(19) United States
(12) Patent Application Publication
(10) Pub. No.: US 2020/0054309 A1
(43) Pub. Date: Feb. 20, 2020

(54) JAW ASSEMBLY FOR SURGICAL TOOL AND BIOPSY SAMPLE COLLECTION MEMBER

(71) Applicant: Jacek KRZYZANOWSKI, Etobicoke (CA)

(72) Inventor: Jacek KRZYZANOWSKI, Etobicoke (CA)

(21) Appl. No.: 16/486,299

(22) PCT Filed: Feb. 21, 2018
(86) PCT No.: PCT/CA2018/050194

§ 371 Date: Aug. 15, 2019

Related U.S. Application Data
(60) Provisional application No. 62/462,021, filed on Feb. 22, 2017.
(60) Provisional application No. 62/550,860, filed on Aug. 28, 2017.

(51) Int. Cl. A61B 10/06; A61B 10/04

(52) U.S. Cl. CPC A61B 10/06; A61B 2017/2936; A61B 10/04

(57) ABSTRACT

Biopsy jaw assemblies for biopsy forceps devices and other tools are disclosed. In some embodiments, the jaw assemblies comprise first and second jaws pivotally movable between respective open and closed positions. One or more guides are configured to urge one or both jaws toward their respective closed positions to enhance the clamping force of the jaws. In some embodiments of biopsy forceps devices, the first and second jaws are configured to promote adhesion of the one or more biopsy samples to one of the jaws. A collection member comprising an adherent portion for collecting the one or more biopsy samples from the biopsy forceps device is also disclosed.

### CROSS REFERENCE TO RELATED APPLICATIONS AND CLAIM OF PRIORITY

The present application claims priority to U.S. provisional patent application No. 62/462,021 filed on Feb. 22, 2017 and to U.S. provisional patent application No. 62/550,860 filed on Aug. 28, 2017, the entire contents of which are hereby incorporated by reference.

### TECHNICAL FIELD

The disclosure relates generally to medical instruments, and more particularly to jaw assemblies for surgical tools and biopsy sample collection members.

### BACKGROUND OF THE ART

Devices for harvesting biopsy samples from within a patient are commonly used in conjunction with endoscopes. An example of such devices is a biopsy forceps which can be inserted into a working channel of an endoscope. The use of such forceps typically includes a physician locating a tip of the endoscope at the desired location within the body of a patient, advancing the biopsy forceps in the working channel of the endoscope until it protrudes out of the tip of the endoscope, opening jaws of the biopsy forceps, advancing the jaws against the tissue and then closing the jaws to avulse or cut away a biopsy sample from the tissue. The biopsy forceps is then retrieved, while closed, from the endoscope by withdrawing it from the working channel of the endoscope and the biopsy sample is removed from the jaws for in situ fixation. The biopsy sample is then sent for further processing and histopathological evaluation.

It is often desired to obtain more than one biopsy sample during a procedure. For example, in cases of patients with Irritable Bowel Disease (IBD) the current state of the art calls for a minimum of 32 biopsies of the colon to be taken. In these cases, the procedure can be time consuming as the biopsy forceps is repeatedly inserted into and withdrawn from the patient via the working channel of the endoscope to harvest each biopsy sample individually. Furthermore, maintaining the tip of the endoscope in a steady position during the removal and re-insertion of the forceps is often a tedious and difficult task. Accordingly, the clinician's ability to keep track of which areas have been biopsied and which have not can be impaired. Harvesting of multiple biopsy samples from a patient requires the location and/or harvesting sequence of the biopsy samples to be tracked. Accordingly, the physician and/or other personnel involved with the harvesting, processing and/or examination of the biopsy samples must be careful to avoid mixing-up the biopsy samples.

Improvement is desirable.

### SUMMARY

In one aspect, the disclosure describes a jaw assembly for a biopsy forceps device. The assembly comprises:

first and second jaws pivotable about a common pivot axis, the first and second jaws being pivotally movable between respective open and closed positions and configured to cooperatively harvest and retain one or more biopsy samples by actuation of the first and second jaws between their respective open and closed positions;

one or more actuation members for actuating the first and second jaws between their respective open and closed positions; and

one or more guides configured to urge one or more of the first and second jaws toward their respective closed positions when the one or more jaws are actuated toward their respective closed positions using the one or more actuation members.

The one or more actuation members may be configured to cause translation movement of the first and second jaws relative to the one or more guides.

The one or more actuation members may be configured to cause movement of the first and second jaws toward the one or more guides when the first and second jaws are actuated toward their respective closed positions.

The one or more actuation members may comprise a common actuation wire configured to actuate both the first and second jaws.

The one or more actuation members may comprise respective actuation wires for actuating the first and second jaws.

The actuation wires may be configured to be actuated in unison to cause opening and closing of the first and second jaws.

The actuation wires may be connected to respective ones of the first and second jaws at respective offset distances from the common pivot axis.

The one or more guides may comprise an aperture into which a portion of the first and second jaws is progressively received during closing of the first and second jaws.

The aperture may be defined by an urging surface configured to engage with respective radially-outer surfaces of the first and second jaws during closing of the first and second jaws.

The one or more guides may comprise a sleeve.

The first and second jaws may cooperatively define a sample-retaining volume configured to retain a plurality of biopsy samples in an order of harvest.

The first and second jaws may comprise respective inner surfaces cooperatively defining a sample-retaining volume configured to retain the one or more biopsy samples. The inner surface of the second jaw may provide a larger contact area for interfacing with the one or more biopsy samples than the inner surface of the first jaw, to promote adhesion of the one or more biopsy samples to the inner surface of the second jaw.

The one or more guides may comprise a sleeve into which a portion of the first and second jaws is progressively received during closing of the first and second jaws.

The first and second jaws may be configured so that at least a majority of the sample-retaining volume remains outside of the sleeve when the first and second jaws are closed.

The first and second jaws may be pivotally coupled to a common pivot member.

The first and second jaws may be substantially rigid.

In some embodiments, the one or more guides may comprise a sleeve; the first and second jaws may be pivotally coupled to a common pivot member; opposite ends of the common pivot member may be engaged with the sleeve; and the common pivot member may be translatable relative to the sleeve during closing of the first and second jaws.

The sleeve may comprise diametrically opposed slots engaging respective opposite ends of the common pivot member.

In some embodiments, the first and second jaws may define a sample-retaining volume having an elongated shape having a central longitudinal axis; and the one or more guides may define a maximum radially-outer dimension that is the same or less than a maximum radially-outer dimension defined by the first and second jaws when the first and second jaws are closed.

In some embodiments, the one or more guides may comprise a sleeve having a central axis; the first and second jaws may each comprise a radially-outer surface and a transition surface portion extending between two regions of the radially-outer surface having different radially-outer dimensions relative to the central axis of the sleeve; and the sleeve may comprise one or more urging surfaces configured to engage with the transition surface portions of the first and second jaws during closing of the first and second jaws.

In some embodiments, the one or more guides may comprise a sleeve having a central axis; the first and second jaws may each comprise a radially-outer surface and a transition surface portion extending radially inwardly from the radially-outer surface relative to the central axis of the sleeve; and the sleeve may comprise one or more urging surfaces configured to engage with the transition surface portions of the first and second jaws during closing of the first and second jaws.

The transition surface portions may be substantially transverse to the central axis of the sleeve.

The transition surface portions may be substantially oblique to the central axis of the sleeve.

The common pivot axis may have a variable position relative to the one or more guides.

The first jaw may have a first rocking surface for interfacing with a second rocking surface of the second jaw during pivotal movement of the first and second jaws.

The common pivot axis may be disposed at an interface between the first rocking surface and the second rocking surface.

The first and second jaws may be slidingly engaged to respective parallel guide pins. The guide pins may be secured to the one or more guides.

The first and second jaws may be slidingly engaged to a common guide pin.

The common guide pin may have a rectangular transverse cross-sectional profile.

A longitudinal axis of the common guide pin may be transverse to the common pivot axis.

In some embodiments, the one or more guides may comprise a sleeve having a central axis; the first and second jaws may be slidingly engaged to a common guide pin; the common guide pin may have has a rectangular transverse cross-sectional profile; and a dimension of the transverse cross-sectional profile along the central axis of the sleeve may be greater than a dimension of the transverse cross-sectional profile transverse to the central axis of the sleeve.

The common guide pin may be secured to the one or more guides.

In some embodiments, the one or more guides may comprise a sleeve having a central axis; and the assembly may comprise a guide insert for guiding the movement of the first and second jaws. The guide insert may be disposed inside of the sleeve. The guide insert may comprise one or more resilient features for engagement with one or more corresponding sleeve features and for retention of the guide insert inside of the sleeve. The one or more resilient features may be resiliently movable in a radially inward direction relative to the central axis.

The one or more sleeve features may comprise respective receptacles formed in the sleeve for receiving the corresponding one or more resilient features of the guide insert. The one or more resilient features of the guide insert may extend radially outwardly into the corresponding one or more receptacles.

The guide insert may comprise one or more cutouts to facilitate resilient radially inward movement of the one or more resilient features and thereby facilitate insertion of the guide insert into the sleeve during assembly.

The guide insert may comprise one or more stoppers for limiting an opening movement of one or more of the first and second jaws.

The guide insert may comprise a surface defining a proximal end of a sample-retaining volume cooperatively defined by the first and second jaws.

Embodiments may include combinations of the above features.

In another aspect, the disclosure describes a jaw assembly for an endoscopic surgical tool. The assembly comprises:

first and second jaws pivotable about a common pivot axis, the first and second jaws being pivotally movable between respective open and closed positions;

one or more actuation members for actuating the first and second jaws between their respective open and closed positions; and

one or more guides configured to urge one or more of the first and second jaws toward their respective closed positions when the one or more jaws are actuated toward their respective closed positions using the one or more actuation members.

The one or more actuation members may be configured to cause translation movement of the first and second jaws relative to the one or more guides.

The one or more actuation members may be configured to cause movement of the first and second jaws toward the one or more guides when the first and second jaws are actuated toward their respective closed positions.

The one or more actuation members may comprise a common actuation wire configured to actuate both the first and second jaws.

The one or more actuation members may comprise respective actuation wires for actuating the first and second jaws.

The actuation wires may be configured to be actuated in unison to cause opening and closing of the first and second jaws.

The actuation wires may be connected to respective ones of the first and second jaws at respective offset distances from the common pivot axis.

The one or more guides comprise an aperture into which a portion of the first and second jaws is progressively received during closing of the first and second jaws.

The aperture may be defined by an urging surface configured to engage with respective radially-outer surfaces of the first and second jaws during closing of the first and second jaws.

The one or more guides may comprise a sleeve.

The first and second jaws may be pivotally coupled to a common pivot member.

The first and second jaws may be substantially rigid.

In some embodiments, the one or more guides may comprise a sleeve; the first and second jaws may be pivotally coupled to a common pivot member; opposite ends of the common pivot member may be engaged with the sleeve; and the common pivot member may be translatable relative to the sleeve during closing of the first and second jaws.

The sleeve may comprise diametrically opposed slots engaging respective opposite ends of the common pivot member.

In some embodiments, the one or more guides may comprise a sleeve having a central axis; the first and second jaws may each comprise a radially-outer surface and a transition surface portion extending between two regions of the radially-outer surface having different radially-outer dimensions relative to the central axis of the sleeve; and the sleeve may comprise one or more urging surfaces configured to engage with the transition surface portions of the first and second jaws during closing of the first and second jaws.

In some embodiments, the one or more guides may comprise a sleeve having a central axis; the first and second jaws may each comprise a radially-outer surface and a transition surface portion extending radially inwardly from the radially-outer surface relative to the central axis of the sleeve; and the sleeve may comprise one or more urging surfaces configured to engage with the transition surface portions of the first and second jaws during closing of the first and second jaws.

The transition surface portions may be substantially transverse to the central axis of the sleeve.

The transition surface portions may substantially oblique to the central axis of the sleeve.

The common pivot axis may have a variable position relative to the one or more guides.

The first jaw may have a first rocking surface for interfacing with a second rocking surface of the second jaw during pivotal movement of the first and second jaws.

The common pivot axis may be disposed at an interface between the first rocking surface and the second rocking surface.

The first and second jaws may be slidingly engaged to respective parallel guide pins.

The guide pins may be secured to the one or more guides.

The first and second jaws may be slidingly engaged to a common guide pin.

The common guide pin may have a rectangular transverse cross-sectional profile.

A longitudinal axis of the common guide pin may be transverse to the common pivot axis.

In some embodiments, the one or more guides may comprise a sleeve having a central axis; the first and second jaws may be slidingly engaged to a common guide pin; the common guide pin may have a rectangular transverse cross-sectional profile where a dimension of the transverse cross-sectional profile along the central axis of the sleeve is greater than a dimension of the transverse cross-sectional profile transverse to the central axis of the sleeve.

The common guide pin may be secured to the one or more guides.

In some embodiments, the one or more guides may comprise a sleeve having a central axis; and the assembly may comprise a guide insert for guiding the movement of the first and second jaws. The guide insert may be disposed inside of the sleeve. The guide insert may comprise one or more resilient features for engagement with one or more corresponding sleeve features and for retention of the guide insert inside of the sleeve. The one or more resilient features may be resiliently movable in a radially inward direction relative to the central axis of the sleeve.

The one or more sleeve features may comprise respective receptacles formed in the sleeve for receiving the corresponding one or more resilient features of the guide insert. The one or more resilient features of the guide insert may extend radially outwardly into the corresponding one or more receptacles.

The guide insert may comprise one or more cutouts to facilitate resilient radially inward movement of the one or more resilient features and thereby facilitate insertion of the guide insert into the sleeve during assembly.

The guide insert may comprise one or more stoppers for limiting an opening movement of one or more of the first and second jaws.

The first and second jaws may each comprise a scissor blade.

Embodiments may include combinations of the above features.

In another aspect, the disclosure describes a jaw assembly for a biopsy forceps device. The assembly comprises: first and second substantially rigid jaws pivotally movable between respective open and closed positions and configured to cooperatively harvest and retain one or more biopsy samples by actuation of the first and second jaws between their respective open and closed positions, the first and second jaws comprising respective inner surfaces cooperatively defining a sample-retaining volume configured to retain the one or more biopsy samples, the inner surface of the second jaw providing a larger contact area for interfacing with the one or more biopsy samples than the inner surface of the first jaw to promote adhesion of the one or more biopsy samples to the inner surface of the second jaw.

The sample-retaining volume may be configured to retain a plurality of biopsy samples.

The sample-retaining volume may be configured to accommodate a row of biopsy samples in an order of harvest.

The sample-retaining volume may be configured to accommodate a row of biopsy samples in an orientation of harvest.

The sample-retaining volume may be configured to retain five or more biopsy samples.

The sample-retaining volume may have an elongated shape having a central longitudinal axis.

A contact interface between the first and second jaws may lie entirely in a plane that intersects and that is parallel to the central longitudinal axis.

A face of the second jaw may lie substantially entirely in a plane that intersects and that is parallel to the central longitudinal axis when the second jaw is in its closed position.

A portion of a face of the first jaw may be offset from the plane when the first jaw is in its closed position.

In some embodiments, the first jaw may comprise a window defined in the inner surface of the first jaw; and the second jaw may be free of windows defined in the inner surface of the second jaw.

A fenestration of the first jaw may be different from a fenestration of the second jaw.

The first and second jaws may be pivotable about a common pivot axis.

The assembly may comprise one or more guides configured to urge the one or more of the first and second jaws toward their respective closed positions when the one or more jaws are actuated toward their respective closed positions.

Embodiments may include combinations of the above features.

In a further aspect, the disclosure describes a kit for harvesting one or more biopsy samples. The kit comprises:

a biopsy forceps device comprising first and second jaws configured to cooperatively harvest and retain one or more biopsy samples; and

a collection member comprising an adherent portion configured to contact and adhere to the one or more biopsy samples being retained by the biopsy forceps device, and collect the one or more biopsy samples from the biopsy forceps device upon withdrawal of the collection member and the biopsy forceps device from each other.

The biopsy forceps device may be configured to retain all of the one or more biopsy samples in only one of the first and second jaws.

The adherent portion may be disposed exclusively on one side of the collection member.

The adherent portion may comprise a chemical bonding agent.

The chemical bonding agent may comprise an epoxy.

The chemical bonding agent may comprise glue.

The adherent portion may comprise a mechanical bonding agent.

The mechanical bonding agent may comprise a surface texture.

The mechanical bonding agent may comprise an array of sample-adhering features.

The adherent portion may be configured to cause electrostatic bonding of the one or more biopsy samples to the collection member.

The adherent portion may be configured to cause thermal bonding of the one or more biopsy samples to the collection member.

The adherent portion may be configured to cause freeze bonding of the one or more biopsy samples to the collection member.

The adherent portion may be configured to cause dry bonding of the one or more biopsy samples to the collection member.

The adherent portion may comprise one or more vacuum ports in communication with a vacuum source.

The collection member may be configured to be received between the first and second jaws.

The collection member may comprise a guide for constraining a position of the biopsy forceps device during the collection of the one or more biopsy samples.

The guide may define an opening for receiving the first or second jaw therethrough. The opening may have a shape that matches at least a majority of a transverse cross-sectional profile of the first or second jaw received therethrough for constraining the orientation of the biopsy forceps device.

The collection member may have a tubular configuration where the opening defined by the guide is an opening to a lumen of the tubular collection member in which the first or second jaw is received during the collection of the one or more biopsy samples.

The adherent portion may be disposed on an outer wall of the tubular collection member.

The collection member may comprise a label indicative of an order of harvest of a plurality of the one or more biopsy samples.

In some embodiments, the biopsy forceps device may comprise a sleeve into which a portion of the first and second jaws is progressively received during closing of the first and second jaws, the sleeve urging the first and second jaws toward their respective closed positions when the one or more jaws are actuated toward their respective closed positions; the first and second jaws may cooperatively define a sample-retaining volume configured to retain a plurality of the one or more biopsy samples in an order of harvest; and at least a majority of the sample-retaining volume may remain outside of the sleeve when the first and second jaws are closed.

Embodiments may include combinations of the above features.

In a further aspect, the disclosure describes a collection member for collecting one or more biopsy samples retained by a biopsy forceps device. The collection member comprises:

an adherent portion configured to contact with and adhere to the one or more biopsy samples being retained by the biopsy forceps device, and collect the one or more biopsy samples from the biopsy forceps device upon withdrawal of the collection member and the biopsy forceps device from each other; and

a guide for constraining a disposition of the biopsy forceps device relative to the adherent portion during the collection of the one or more biopsy samples.

The adherent portion may be disposed exclusively on one side of the collection member.

The adherent portion may comprise a chemical bonding agent.

The adherent portion may comprise a mechanical bonding agent.

The guide may define an opening for receiving one of the jaws therethrough.

The opening may have a shape that substantially matches at least a majority of a transverse cross-sectional profile of the one jaw received therethrough for constraining an orientation of the biopsy forceps device.

The collection member may have a tubular configuration where the opening defined by the guide is an opening to a lumen of the tubular collection member in which the one jaw is received during the collection of the one or more biopsy samples.

The adherent portion may be disposed on an outer wall of the tubular collection member.

The collection member may comprise a label indicative of an order of harvest of a plurality of the one or more biopsy samples.

The collection member may be configured to be received between the first and second jaws.

Embodiments may include combinations of the above features.

In a further aspect, the disclosure describes a method for collecting one or more biopsy samples from a biopsy forceps device comprising first and second jaws configured to cooperatively harvest and retain the one or more biopsy samples. The method comprises:

contacting an adherent portion of a collection member with the one or more biopsy samples being retained by the biopsy forceps device;

adhering the one or more biopsy samples to the adherent portion; and collecting the one or more biopsy samples adhered to the adherent portion of the collection member from the biopsy forceps device.

The method may comprise clamping the collection member between the first and second jaws to adhere the one or more biopsy samples to the collection member. The method may comprise unclamping the collection member and then withdrawing the collection member and the biopsy forceps device from each other.

Adhering the one or more biopsy samples to the collection member may comprise chemically bonding the one or more biopsy samples to the collection member.

Adhering the one or more biopsy samples to the collection member may comprise mechanically bonding the one or more biopsy samples to the collection member.

Adhering the one or more biopsy samples to the collection member may comprise electrostatically bonding the one or more biopsy samples to the collection member.

Adhering the one or more biopsy samples to the collection member may comprise bonding the one or more biopsy samples to the collection member using coagulation.

Adhering the one or more biopsy samples to the collection member may comprise bonding the one or more biopsy samples to the collection member using a vacuum source.

Adhering the one or more biopsy samples to the collection member may comprise bonding the one or more biopsy samples to the collection member using an epoxy.

Collecting the one or more biopsy samples may comprise withdrawing the collection member and the biopsy forceps device from each other after opening the first and second jaws of the biopsy forceps device.

Embodiments may include combinations of the above features.

Further details of these and other aspects of the subject matter of this application will be apparent from the detailed description included below and the drawings.

### DESCRIPTION OF THE DRAWINGS

Reference is now made to the accompanying drawings, in which:

FIG. 1A shows a perspective view of an exemplary jaw assembly of a biopsy forceps device according to one embodiment;

FIG. 1B shows a perspective exploded view of the jaw assembly of FIG. 1A;

FIG. 1C shows a side elevation view of the jaw assembly of FIG. 1A in its closed configuration;

FIG. 1D shows an axial cross-sectional view of the jaw assembly of FIG. 1A in its closed configuration taken along line 1D-1D in FIG. 1C;

FIG. 1E shows a cross-sectional view of the jaw assembly of FIG. 1A in its closed configuration taken along line 1E-1E in FIG. 1C;

FIG. 1F shows a side elevation view of the jaw assembly of FIG. 1A in its open configuration;

FIG. 1G shows an axial cross-sectional view of the jaw assembly of FIG. 1A in its open configuration taken along line 1G-1G in FIG. 1F;

FIG. 2A shows a perspective view of an exemplary jaw assembly of a biopsy forceps device according to another embodiment;

FIG. 2B shows an cross-sectional view of the jaw assembly of FIG. 2A in its closed configuration taken along line 2-2 in FIG. 2A;

FIG. 2C shows an axial cross-sectional view of the jaw assembly of FIG. 2A in its open configuration taken along line 2-2 in FIG. 2A;

FIG. 3A shows an axial cross-sectional view of an exemplary jaw assembly of a biopsy forceps device according to another embodiment, in its closed configuration;

FIG. 3B shows an enlarged view of region 3 of the jaw assembly of FIG. 3A;

FIG. 3C shows an axial cross-sectional view of the jaw assembly of FIG. 3A in its open configuration;

FIG. 4A shows a perspective view of an exemplary jaw assembly of a biopsy forceps device according to another embodiment;

FIG. 4B shows a perspective exploded view of the jaw assembly of FIG. 4A;

FIG. 4C shows a side elevation view of the jaw assembly of FIG. 4A in its closed configuration;

FIG. 4D shows an axial cross-sectional view of the jaw assembly of FIG. 4A in its closed configuration taken along line 4D-4D in FIG. 4C;

FIG. 4E shows a cross-sectional view of the jaw assembly of FIG. 4A in its closed configuration taken along line 4E-4E in FIG. 4C;

FIG. 4F shows a side elevation view of the jaw assembly of FIG. 4A in its open configuration;

FIG. 4G shows an axial cross-sectional view of the jaw assembly of FIG. 4A in its open configuration taken along line 4G-4G in FIG. 4F;

FIG. 5A shows a perspective view of an exemplary jaw assembly of a biopsy forceps device according to another embodiment;

FIG. 5B shows a perspective exploded view of the jaw assembly of FIG. 5A;

FIG. 5C shows a side elevation view of the jaw assembly of FIG. 5A in its closed configuration;

FIG. 5D shows an axial cross-sectional view of the jaw assembly of FIG. 5A in its closed configuration taken along line 5D-5D in FIG. 5C;

FIG. 5E shows a cross-sectional view of the jaw assembly of FIG. 5A in its closed configuration taken along line 5E-5E in FIG. 5C;

FIG. 5F shows a side elevation view of the jaw assembly of FIG. 5A in its open configuration;

FIG. 5G shows an axial cross-sectional view of the jaw assembly of FIG. 5A in its open configuration taken along line 5G-5G in FIG. 5F;

FIG. 6A shows a perspective view of an exemplary jaw assembly of a biopsy forceps device according to another embodiment;

FIG. 6B shows a perspective exploded view of the jaw assembly of FIG. 6A;

FIG. 6C shows a side elevation view of the jaw assembly of FIG. 6A in its closed configuration;

FIG. 6D shows an axial cross-sectional view of the jaw assembly of FIG. 6A in its closed configuration taken along line 6D-6D in FIG. 6C;

FIG. 6E shows a cross-sectional view of the jaw assembly of FIG. 6A in its closed configuration taken along line 6E-6E in FIG. 6C;

FIG. 6F shows a side elevation view of the jaw assembly of FIG. 6A in its open configuration;

FIG. 6G shows an axial cross-sectional view of the jaw assembly of FIG. 6A in its open configuration taken along line 6G-6G in FIG. 6F;

FIG. 7A shows a perspective view of an exemplary jaw assembly of a biopsy forceps device according to another embodiment;

FIG. 7B shows a perspective exploded view of the jaw assembly of FIG. 7A;

FIG. 7C shows a side elevation view of the jaw assembly of FIG. 7A in its closed configuration;

FIG. 7D shows an axial cross-sectional view of the jaw assembly of FIG. 7A in its closed configuration taken along line 7D-7D in FIG. 7C;

FIG. 7E shows a cross-sectional view of the jaw assembly of FIG. 7A in its closed configuration taken along line 7E-7E in FIG. 7C;

FIG. 7F shows a side elevation view of the jaw assembly of FIG. 7A in its open configuration;

FIG. 7G shows an axial cross-sectional view of the jaw assembly of FIG. 7A in its open configuration taken along line 7G-7G in FIG. 7F;

FIG. 8A shows a perspective view of an exemplary jaw assembly of a biopsy forceps device according to another embodiment;

FIG. 8B shows a perspective exploded view of the jaw assembly of FIG. 8A;

FIG. 8C shows a side elevation view of the jaw assembly of FIG. 8A in its closed configuration;

FIG. 8D shows an axial cross-sectional view of the jaw assembly of FIG. 8A in its closed configuration taken along line 8D-8D in FIG. 8C;

FIG. 8E shows a cross-sectional view of the jaw assembly of FIG. 8A in its closed configuration taken along line 8E-8E in FIG. 8C;

FIG. 8F shows a side elevation view of the jaw assembly of FIG. 8A in its open configuration;

FIG. 8G shows an axial cross-sectional view of the jaw assembly of FIG. 8A in its open configuration taken along line 8G-8G in FIG. 8F;

FIG. 9A shows an axial cross-sectional view of an exemplary jaw assembly of a biopsy forceps device according to another embodiment, in its closed configuration;

FIGS. 9B-9E show enlarged views of region 9 of the jaw assembly of FIG. 9A according to different embodiments;

FIG. 9F shows an axial cross-sectional view of the jaw assembly of FIG. 9A in its open configuration;

FIG. 10A shows a perspective view of an exemplary jaw assembly of a biopsy forceps device according to another embodiment;

FIG. 10B shows a perspective exploded view of the jaw assembly of FIG. 10A;

FIG. 10C shows a side elevation view of the jaw assembly of FIG. 10A in its closed configuration;

FIG. 10D shows an axial cross-sectional view of the jaw assembly of FIG. 10A in its closed configuration taken along line 10D-10D in FIG. 100;

FIG. 10E shows a cross-sectional view of the jaw assembly of FIG. 10A in its closed configuration taken along line 10E-10E in FIG. 10C;

FIG. 10F shows a side elevation view of the jaw assembly of FIG. 10A in its open configuration;

FIG. 10G shows an axial cross-sectional view of the jaw assembly of FIG. 10A in its open configuration taken along line 10G-10G in FIG. 10F;

FIG. 11A shows a perspective view of an exemplary jaw assembly of a biopsy forceps device according to another embodiment;

FIG. 11B shows a perspective exploded view of the jaw assembly of FIG. 11A;

FIG. 11C shows a side elevation view of the jaw assembly of FIG. 11A in its closed configuration;

FIG. 11D shows an axial cross-sectional view of the jaw assembly of FIG. 11A in its closed configuration taken along line 11D-11D in FIG. 11C;

FIG. 11E shows a cross-sectional view of the jaw assembly of FIG. 11A in its closed configuration taken along line 11E-11E in FIG. 11C;

FIG. 11F shows a side elevation view of the jaw assembly of FIG. 11A in its open configuration;

FIG. 11G shows an axial cross-sectional view of the jaw assembly of FIG. 11A in its open configuration taken along line 11G-11G in FIG. 11F;

FIG. 12A shows a perspective view of an exemplary jaw assembly of a biopsy forceps device according to another embodiment;

FIG. 12B shows a perspective exploded view of the jaw assembly of FIG. 12A;

FIG. 12C shows a side elevation view of the jaw assembly of FIG. 12A in its closed configuration;

FIG. 12D shows an axial cross-sectional view of the jaw assembly of FIG. 12A in its closed configuration taken along line 12D-12D in FIG. 12C;

FIG. 12E shows a cross-sectional view of the jaw assembly of FIG. 12A in its closed configuration taken along line 12E-12E in FIG. 12C;

FIG. 12F shows a side elevation view of the jaw assembly of FIG. 12A in its open configuration;

FIG. 12G shows an axial cross-sectional view of the jaw assembly of FIG. 12A in its open configuration taken along line 12G-12G in FIG. 12F;

FIG. 12H shows a perspective view of a guide insert of the jaw assembly of FIG. 12A;

FIG. 12I shows a bottom view of the guide insert of FIG. 12H;

FIG. 12J shows a front elevation view of the guide insert of FIG. 12H;

FIG. 13A shows a perspective view of an exemplary jaw assembly of a endoscopic surgical tool;

FIG. 13B shows a perspective exploded view of the jaw assembly of FIG. 13A;

FIG. 13C shows an axial cross-sectional view of the jaw assembly of FIG. 13A in its closed configuration;

FIG. 13D shows an axial cross-sectional view of the jaw assembly of FIG. 13D in its open configuration;

FIG. 14A shows a graph of a clamping force versus the angle of opening of the jaw assembly of FIG. 1A;

FIG. 14B shows an enlarged view of the jaw assembly of FIG. 1A with a sleeve of the jaw assembly being partially cut away;

FIGS. 15A and 15B show perspective views of a kit for harvesting one or more biopsy samples where the kit comprises a biopsy forceps device and a collection member;

FIGS. 16A-16C graphically illustrate a method for collecting biopsy samples from a biopsy forceps device using the collection member of FIGS. 15A and 15B, where the biopsy samples are retained in an upper jaw of the biopsy forceps device;

FIGS. 17A-17C graphically illustrate a method for collecting biopsy samples from a biopsy forceps device using the collection member of FIGS. 15A and 15B, where the biopsy samples are retained in a lower jaw of the biopsy forceps device;

and

FIGS. 18A and 18B are perspective views of another kit for harvesting one or more biopsy samples where the kit comprises a biopsy forceps device and a collection member according to another embodiment;

FIGS. 19A-19C graphically illustrate a method for collecting biopsy samples from a biopsy forceps device using the collection member of FIGS. 18A and 18B, where the biopsy samples are retained in an upper jaw of the biopsy forceps device;

FIG. 20 is a perspective view of the collection member of the kit of FIGS. 18A and 18B showing an exemplary label indicative of an order of harvest of the biopsy sample;

FIGS. 21A-21D graphically illustrate another method for collecting biopsy samples from a biopsy forceps device using a collection member; and

FIG. 22 shows a flowchart of a method for collecting one or more biopsy samples from a biopsy forceps device.

### DETAILED DESCRIPTION

The following disclosure relates to jaw assemblies for biopsy forceps devices configured to harvest and retain one or more biopsy samples and for other tools. In some embodiments, the jaw assemblies disclosed herein are configured to harvest and retain a plurality of biopsy samples in the order of harvest so that the repeated insertion and withdrawal of the biopsy forceps device into and out of the patient may be reduced. In some embodiments, the jaw assemblies disclosed herein are configured to also retain the plurality of biopsy samples in the orientation of harvest. In some embodiments, the jaw assemblies may comprise a guide member that is configured to enhance the clamping force (e.g., biting action) of the jaws and facilitate the harvesting of the biopsy samples. In some embodiments, the jaws of the jaw assemblies may be configured to promote the adhesion of the biopsy samples harvested to one of the jaws. This may facilitate the retention of the biopsy samples in the biopsy forceps device during use and also facilitate the collection of the biopsy samples from the biopsy forceps device.

Aspects of this disclosure are applicable to other (e.g., endoscopic and/or surgical) tools that can benefit from an enhanced clamping or closing force. For example, the term “jaws” as used herein is intended to encompass two or more opposable parts capable opening and closing for holding, crushing, avulsing and/or cutting something between them. For example, aspects of this disclosure can be applicable to other tools such as surgical clip appliers, scissors, grabbers, retrievers, suturing devices, punches and any combination of these tools.

Also disclosed is an adherent collection member that facilitates the collection of the biopsy sample(s) from biopsy forceps devices and, in the case of multi-sample biopsy forceps devices, facilitates the tracking of the order and orientation of collection of the biopsy samples and cataloguing of the biopsy samples according to the location from which each biopsy sample is taken.

Aspects of various embodiments are described through reference to the drawings.

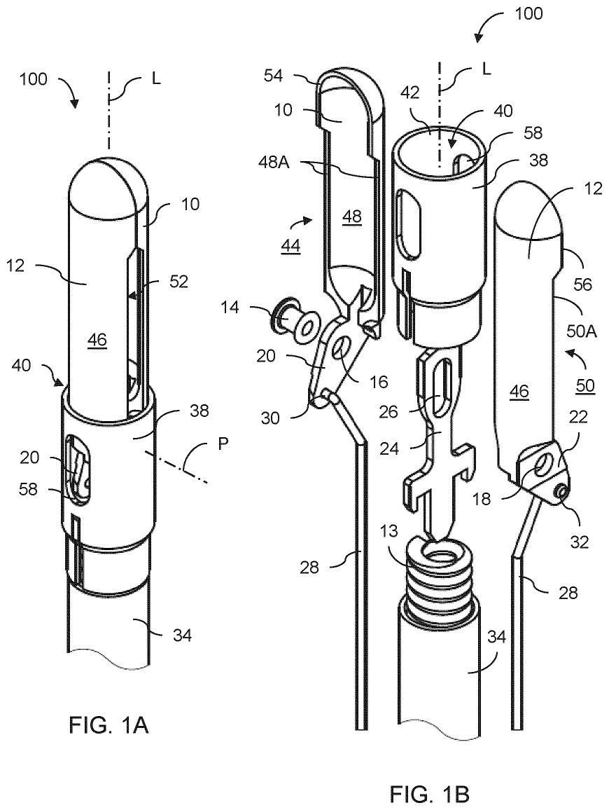

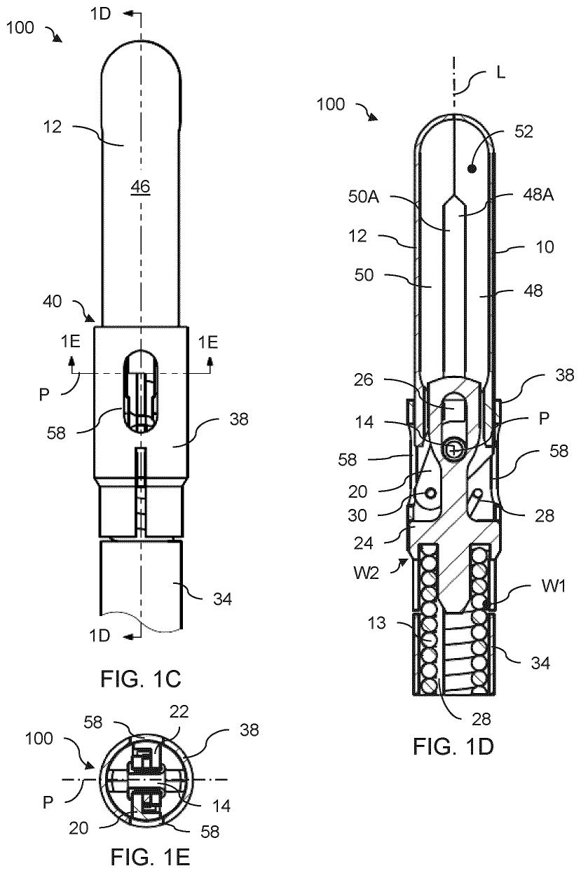

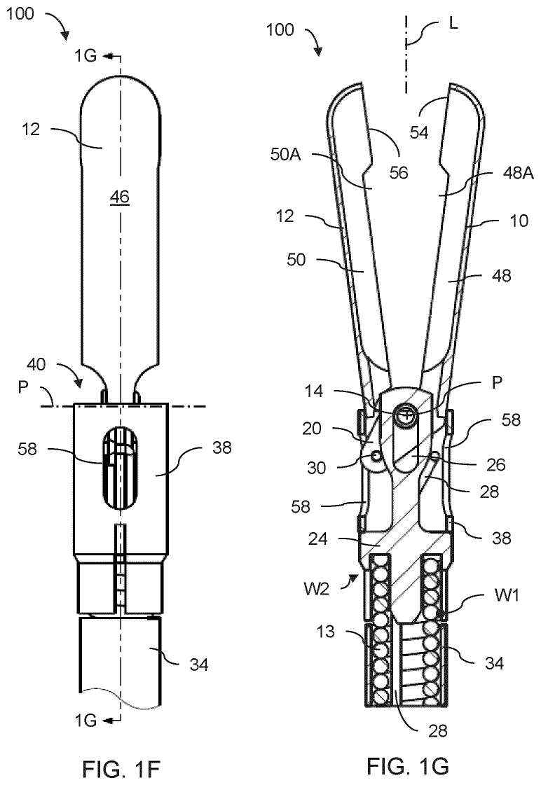

FIGS. 1A-1G illustrate an exemplary embodiment of a biopsy jaw assembly 100 for a biopsy forceps device, which may be inserted into a working channel of an endoscope (e.g., flexible or rigid) or other tubular member for the purpose of harvesting one or more biopsy samples from a patient. Biopsy jaw assembly 100 may be configured for frontal tissue sampling. In reference to FIGS. 1A-1G, jaw assembly 100 may comprise first jaw 10 and second jaw 12 pivotally movable between respective open and closed positions (e.g., see FIGS. 1D and 1G respectively) and configured to cooperatively harvest and retain one or more biopsy samples by actuation of first jaw 10 and second jaw 12 between their respective open and closed positions.

The terms “proximal” and “distal” are used herein to describe opposite directions along central longitudinal axis “L”. The term “proximal” is used to describe a direction toward an operator of jaw assembly 100 (i.e., downward along axis L in FIG. 1A) and the term “distal” is used to describe a direction away from the operator of jaw assembly 100 (i.e., upward along axis L in FIG. 1A).

Jaw assembly 100 may be attached to a distal end of an elongated tube such as shaft 13, which may be flexible or rigid in various embodiments. Jaw assembly 100 may be opened and closed by a suitable actuating mechanism as described below. Such actuating mechanism may be controlled by manipulating suitable controls located at a proximal end of shaft 13. The controls may be configured to permit actuation and/or rotation of jaw assembly 100 by a physician or an assistant during operation for example.

It is understood that biopsy forceps devices comprising the jaw assemblies disclosed herein may be used in conjunction with an endoscope with or without utilizing a working channel thereof. It is also understood that such biopsy forceps devices may be suitable for use without an endoscope, for instance for a cardiac muscle biopsy. In various embodiments, the jaw assemblies disclosed herein may be suitable for use at sites (e.g., colon, urinary tract, reproductive organs, cardiac tissue, or the like) deep within the body of a patient. For example, such biopsy forceps device may be configured to be passed through an introducer or guiding catheter. In some embodiments, the biopsy forceps devices disclosed herein may be provided with electrical connections for cautery or hot biopsy applications. In some embodiments, biopsy forceps devices as disclosed herein may be suitable for veterinary procedures.

In some embodiments, first jaw 10 and second jaw 12 may be pivotable (i.e., hinged) about a common pivot axis P. For example first jaw 10 and second jaw 12 may be pivotally coupled using a common pivot member 14 (e.g., pin or eyelet) via holes 16, 18 formed in respective arms 20, 22 of first jaw 10 and second jaw 12 respectively. First jaw 10 and second jaw 12 may be slidingly engaged with flat member 24 via slot 26. For example, arm 20 of first jaw 10 may be disposed on one side of flat member 24 and arm 22 of second jaw 12 may be disposed on an opposite side of flat member 24. Arms 20, 22 may be pivotally secured via pivot member 14 extending through flat member 24 via holes 16, 18 and slot 26. Slot 26 may be oriented along longitudinal axis L of jaw assembly 100 in order to permit longitudinal (i.e., distal and proximal) movement of first jaw 10 and second jaw 12 relative to flat member 24 during the actuation (i.e., opening and closing) of first jaw 10 and second jaw 12. Accordingly, common pivot axis P may move along longitudinal axis L due to movement of pin 14 within slot 26 during actuation.

Jaw assembly 100 may comprise one or more actuation members for actuating first jaw 10 and second jaw 12 between their respective open and closed positions. For example, as illustrated, jaw assembly 100 may comprise respective actuation wires 28 for actuating first jaw 10 and second jaw 12. In some embodiments, one or both wires 28 may have a multi-strand construction. In some embodiments, one or both wires 28 may comprise a solid wire.

Actuation wires 28 may be connected to respective ones of first jaw 10 and second jaw 12 at actuation points 30 and 32 respectively disposed on arms 20 and 22 at an offset distance from common pivot axis P. The actuation (i.e., pulling or pushing) of actuation wires 28 along longitudinal axis L of jaw assembly 100 may cause the opening and closing of first jaw 10 and of second jaw 12. For example, the pushing (i.e., distal movement) of both actuation wires 28 in unison may cause pivot member 14 to slide distally in slot 26 and cause first jaw 10 and second jaw 12 to pivot about common pivot axis P in respective opposite directions that cause opening of first jaw 10 and second jaw 12. Alternatively, the pulling (i.e., proximal movement) of both actuation wires 28 in unison may cause pivot member 14 to slide proximally in slot 26 and cause first jaw 10 and second jaw 12 to pivot about common pivot axis P in respective opposite directions that cause closing of first jaw 10 and second jaw 12.

Shaft 13 may comprise an opening extending therethrough along longitudinal axis L of jaw assembly 100 for receiving therein part of flat member 24. Actuation wires 28 may also extend through shaft 13 and to a suitable actuation handle or other control(s) (e.g., user interface) for pulling and pushing on actuation wires 28 in unison. Alternatively, actuating wires 28 may terminate within shaft 13 and be operatively connected to hydraulic, pneumatic, electric or other actuators contained within shaft 13 for example. In some embodiments, shaft 13 may be sufficiently flexible to facilitate lateral deflexion of the biopsy forceps device in order to facilitate the channelling of the biopsy forceps device along a curved path, which may be relatively long and/or tortuous, defined by a working channel of an endoscope for example. In some embodiments, shaft 13 may comprise a coil spring made of a suitable metallic material. In some embodiments, shaft 13 may, for example be of a type as disclosed in U.S. Pat. No. 6,309,404, which is incorporated herein by reference. Flexible sheath 34 may extend over shaft 13 and actuation wires 28 may extend proximally through shaft 13 and flexible sheath 34 to suitable control means. Flexible sheath 34 may be made of a suitable plastic material. Flexible sheath 34 may be secured to shaft 13 by suitable means.

Jaw assembly 100 may comprise one or more guides configured to urge first jaw 10 and/or second jaw 12 toward their respective closed positions when first jaw 10 and/or second jaw 12 is/are actuated toward their respective closed positions using actuation wires 28. The guide(s) may have the form of sleeve 38 as illustrated herein but it is understood that such guide(s) may have different forms suitable for enhancing the closing moment of first jaw 10 and/or second jaw 12 in order to enhance the cutting performance of jaw assembly 100 for example. In some embodiments, such guide(s) may comprise any suitable arrangement of one or more shoulder surfaces configured to supplement the closing moment applied to first jaw 10 and/or second jaw 12 by actuation wires 28 alone.

Actuation wires 28 may be configured to cause relative translation movement between jaws 10, 12 and the guide(s) (e.g., sleeve 38). For example, in some embodiments, sleeve 38 may comprise aperture 40 extending through sleeve 38 and into which a portion of first jaw 10 and second jaw 12 is progressively received during closing of first jaw 10 and of second jaw 12. For example, arms 20, 22 of first jaw 10 and second jaw 12 respectively may be disposed inside sleeve 38, and jaws 10, 12 may be longitudinally movable within sleeve 38 by the longitudinal movement afforded by the movement of pivot member 14 within slot 26. As first jaw 10 and second jaw 12 are being opened, first jaw 10 and second jaw 12 may translate distally relative to sleeve 38. Alternatively, as first jaw 10 and second jaw 12 are being closed, first jaw 10 and second jaw 12 may translate proximally relative to sleeve 38. Common pivot axis P may have a position that is variable relative to sleeve 38 by way of movement of pivot member 14 in slot 26 when first and second jaws 10, 12 are actuated.

Sleeve 38 may define one or more urging surfaces 42 configured to engage with one or more radially-outer surfaces 44, 46 of first jaw 10 and second jaw 12 respectively when first jaw 10 and second jaw 12 are actuated. In some embodiments, urging surface 42 may be a radially-inner surface of sleeve 38. For example, in some embodiments, aperture 40 in sleeve 38 may be defined by such radially-inner urging surface 42 of sleeve 38. Urging surface 42 may be disposed at or near a distal end of sleeve 38. For example, urging surface 42 may be part of a distal rim or edge of sleeve 38. In various embodiments, urging surface 42 may comprise a squared distal end surface of sleeve 38 and/or urging surface 42 may comprise a chamfered, bevelled, and/or rounded surface at or near the distal end of sleeve 38.

When first jaw 10 and second jaw 12 are pulled proximally into sleeve 38 via actuation wires 28 from the open configuration shown in FIG. 1G toward the closed configuration shown in FIG. 1D, urging surface 42 of sleeve 38 may engage respective radially-outer surfaces 44, 46 of first jaw 10 and second jaw 12 in order to urge first jaw 10 and second jaw 12 toward their respective closed positions. The frictional/sliding engagement of urging surface 42 of sleeve 38 with the respective radially-outer surfaces 44, 46 of first jaw 10 and second jaw 12 may effectively supplement the closing moment of first jaw 10 and second jaw 12 provided via the forces applied by actuation wires 28 at the respective actuation points 30 and 32 offset from common pivot axis P. For example, urging surface 42 may serve as a shoulder surface that supplements the closing moment of first jaw 10 and second jaw 12 by converting a longitudinal force applied to first jaw 10 and second jaw 12 via actuation wires 28 into respective clamping forces applied directly to radially-outer surfaces 44, 46 of first jaw 10 and second jaw 12 in order to enhance the total clamping force of first jaw 10 and second jaw 12.

In some embodiments, sleeve 38 may have a tubular configuration and may have a substantially circular cross-sectional profile transverse to longitudinal axis L. Sleeve 38 may have a central axis that is substantially coaxial with longitudinal axis L. In some embodiments, sleeve 38 may have a non-circular (e.g., oval, polygonal) cross-sectional profile transverse to longitudinal axis L. Similarly, radially-outer surfaces 44, 46 of first jaw 10 and second jaw 12 may cooperatively define a transverse cross-sectional profile that is substantially circular, partially circular or non-circular when jaws 10 and 12 are closed.

Urging surface 42 of sleeve 38 may be circumferentially continuous about longitudinal axis L. However, it is understood that urging surface 42 of sleeve 38 or of some other type of guide(s) may not necessarily be circumferentially continuous. For example, such guide may comprise one or more separate urging surfaces 42 respectively associated with each of jaws 10 and 12. It is also understood that aspects of jaw assembly 100 disclosed herein could be used to enhance the clamping force of either both or only one of first jaw 10 and second jaw 12. It is also understood that aspects disclosed herein could be used for enhancing the closing performance of jaw assemblies comprising more than two jaws (e.g., four jaws).

First jaw 10 and second jaw 12 may comprise respective inner surfaces 48, 50 cooperatively defining a sample-retaining volume 52 (see FIG. 1D) configured to retain the one or more biopsy samples harvested by the biopsy forceps device. In some embodiments, sample-retaining volume 52 may be configured to retain a plurality of biopsy samples so that jaw assembly 100 may be part of a multi-sample biopsy forceps device. In some embodiments, sample-retaining volume 52 may be configured to retain five (5) or more biopsy samples. In some embodiments, sample-retaining volume 52 may be configured to retain between five (5) and eight (8) biopsy samples. In some embodiments, sample-retaining volume 52 may be configured to retain eight (8) or more biopsy samples. In some embodiments, a major portion of sample-retaining volume 52 may have a generally cylindrical shape that is substantially coaxial with longitudinal axis L. Accordingly, inner surface 48 of first jaw 10 and inner surface of second jaw 12 may each define part of the cylindrical shape. In some embodiments, a major portion of sample-retaining volume 52 may have a generally tubular shape that is substantially coaxial with longitudinal axis L. The tubular shape may have a generally circular or a non-circular (e.g., polygonal) transverse cross-sectional profile.

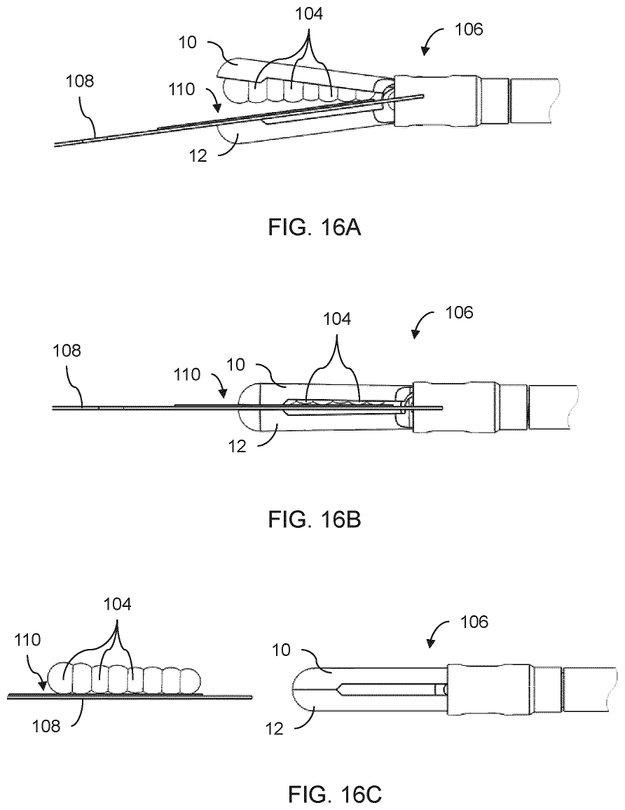

In some embodiments, sample-retaining volume 52 may be configured to accommodate a row of biopsy samples (e.g., see FIG. 16A) in an order and orientation of harvest. For example, sample-retaining volume 52 may have a shape that is elongated along longitudinal axis L. Accordingly, the configuration of jaw assembly 100 may permit the harvesting of a plurality of biopsy samples with a single insertion and removal of the biopsy forceps device in and out of the patient. As the biopsy samples are sequentially harvested using jaw assembly 10, previously harvested biopsy samples retained in sample-retaining volume 52 are sequentially pushed rearwardly (i.e., proximally) into sample-retaining volume 52 so as to form a row of biopsy samples in the order and orientation of harvest. For example, the first biopsy sample harvested in the row would be retained in a proximal/rear region of sample-retaining volume 52 and the last (e.g., eighth) biopsy sample harvested would be retained in a distal/forward region of sample-retaining volume 52. In some embodiments, inner surface 48 of first jaw 10 and/or inner surface 50 of second jaw 12 may be coated with a lubricious substance to aid the sliding movement of biopsy samples proximally in sample-retaining volume 52. In some embodiments, inner surface 48 of first jaw 10 and/or inner surface 50 of second jaw 12 may have a relatively smooth surface finish and may be free of features that may snag the biopsy samples during their retrieval from first and/or second jaw 10, 12.

The harvesting (e.g., avulsing, cutting, clipping) of biopsy samples from the patient may be performed mainly by distal regions of first jaw 10 and second jaw 12 as they are brought together during closing of the jaws 10, 12. Accordingly, one or both of faces 54, 56 of first jaw 10 and second jaw 12 may comprise a cutting edge that facilitates the harvesting of the biopsy sample(s). In some embodiments, one or both of faces 54, 56 may be serrated. Due to the elongated shape of first jaw 10 and second jaw 12 along longitudinal axis L and to the relatively large distance (i.e., moment arm) between the distal ends of jaws 10, 12 from pivot member 14, a relatively large closing moment about common pivot axis P may be needed or desired on first jaw 10 and/or second jaw 12 to achieve the desired clamping force at the distal ends of jaws 10, 12 in comparison with a shorter single-sample jaw assembly. Accordingly, the use of sleeve 38 or other type of guide as disclosed herein may be beneficial to the harvesting performance of multi-sample jaw assemblies.

The use of sleeve 38 to supplement the clamping force of jaws 10, 12 may also promote a relatively small diametric or cross-sectional size of jaw assembly 100 while, at the same time, allowing an adequate clamping force to be obtained. For given actuation forces provided by actuation wires 28, instead of achieving the desired actuation moment using large offset distances of actuation points 30, 32 from common pivot axis P, sleeve 38 serves to supplement the closing moment obtained from smaller offset distances by converting some of the longitudinal actuation forces provided by actuation wires 28 into respective clamping forces onto jaws 10, 12. In other words, for given longitudinal actuation forces provided by actuation wires 28, the use of sleeve 38 may allow, in some embodiments, increasing of the total clamping forces on jaws 10 and/or 12 without increasing the offset distances of actuation points 30, 32 from common pivot axis P and hence without having to significantly increase the girth of the biopsy forceps device.

As explained above, the closing of first jaw 10 and second jaw 12 may cause jaws 10, 12 to be translated proximally relative to (stationary) sleeve 38 so that portions of first jaw 10 and second jaw 12 may be progressively received into aperture 40 of sleeve 38. However, in some embodiments, the amount of such proximal translation may be relatively small so that the movement of jaws 10, 12 away from the tissue during closing of jaws 10, 12 may not significantly affect the harvesting performance of jaw assembly 100. For example, in some embodiments, the amount of such proximal translation exhibited between the open and closed configurations may be about 1.3 mm.

In reference to FIG. 1D, first jaw 10 and second jaw 12 may be configured so that sample-retaining volume 52 may remain mostly or entirely outside (axially distal) of sleeve 38 when jaw assembly 100 is in its closed configuration despite the proximal translation movement of first jaw 10 and second jaw 12 during closing. For example, in some embodiments, at least a majority of sample-retaining volume 52 may remain outside of sleeve 38 when jaw assembly 100 is in its closed configuration. This feature may facilitate the retrieval of the biopsy samples from jaw assembly 100 using an adherent collection member as described below.

The configuration of first jaw 10 and second jaw 12 may also reduce or eliminate the likelihood of damaging and/or contaminating the biopsy samples during the harvesting and retaining of the biopsy samples. For example, in some embodiments, jaw assembly 100 may not require the use of a spike with a barb or other means to mechanically anchor (e.g., pierce, hook, skewer) the biopsy sample(s) when retained in first jaw 10 and/or second jaw 12. Also, jaw assembly 100 may, in some embodiments, not require the use of suction, irrigation, pushing mechanisms or storage containers for collecting individual biopsy samples. Further, jaw assembly 100 may, in some embodiments, not require the use of a sleeve moving over substantially the entire length of the jaws (or alternatively jaws moving similarly into such sleeve) and that could potentially damage or alter the harvest orientation of the biopsy samples. Accordingly, jaw assembly 100 may, in some embodiments, promote maintaining the integrity of the biopsy samples for the subsequent laboratory analysis.

In some embodiments, first jaw 10 and second jaw 12 may have a substantially identical configuration. In some embodiments, first jaw 10 and second jaw 12 may be substantially symmetric across a mid-plane that intersects and that is parallel to longitudinal axis L. For example, a contact interface between first jaw 10 and second jaw 12, defined by face 54 of first jaw 10 and face 56 of second jaw 12, may lie entirely in such mid-plane. In some embodiments, a distal portion of face 54 of first jaw 10 may lie in the mid-plane (in the closed configuration) and a proximal portion of face 54 of first jaw 10 may be offset from the mid-plane in order to form windows 48A to sample-retaining volume 52 in inner surface 48 of first jaw 10. Similarly, in some embodiments, a distal portion of face 56 of second jaw 12 may lie in the mid-plane (in the closed configuration) and a proximal portion of face 56 of first jaw 10 may be offset from the mid-plane in order to form windows 50A to sample-retaining volume 52 in inner surface 50 of second jaw 12.

In some embodiments, the configuration of first and second jaws 10, 12 may also promote the retention of the orientation of harvest of each biopsy sample. For example, the configuration (e.g., orientation and elongated shape) of windows 48A and 50A may help retain the harvest orientation of the biopsy samples as the biopsy samples slide proximally in sample-retaining volume 52 as additional biopsy samples are harvested. For example, the outline of window 48A and/or the outline of window 50A may come in contact engagement with the biopsy samples harvested and hinder the rotation of the biopsy samples while still permitting sliding movement of the biopsy sample proximally in sample-retaining volume 52. It is understood that other elongated features such as ribs or grooves could be provided inside one of jaws 10, 12 to promote the retention of the orientation of harvest of each biopsy sample instead of or in addition to relying on the configuration of windows 48A, 50A.

In reference to FIG. 1D, flat member 24 may be made of a metallic material and may be secured to shaft 13 by suitable means such as welding, soldering, gluing, brazing, crimping, pressing or encasing for example. In some embodiments, flat member 24 may be laser welded to sleeve 38 at location W2. Similarly, sleeve 38 may be made of a metallic material and may be secured to shaft 13 by suitable means such as welding, soldering, gluing or crimping for example. In some embodiments, sleeve 38 may be laser welded to shaft 13 at location W1. In some embodiments, sleeve 38 may comprise slots 58 to accommodate the movement of arms 20 and 22 of jaws 10 and 12.

First jaw 10 and second jaw 12 may be relatively rigid so that their actuation between the open and closed configurations does not cause significant or meaningful deflection of the first jaw 10 and second jaw 12 under normal intended use. For example, each jaw 10, 12 may have a transverse cross-sectional profile (i.e., perpendicular to longitudinal axis L) that defines part (e.g., half) of an annulus. Accordingly, the transverse cross-sectional area of each jaw 10, 12 may have a moment of inertia that is sufficiently high to substantially prevent significant bending or flexing of jaws 10, 12 when they are being closed for harvesting a biopsy sample. First jaw 10 and second jaw 12 may be made from a suitable metallic material. Jaws 10 and 12 may be manufactured by any suitable means such as press work (e.g., stamping from sheet metal), machining from a solid blank, moulding, additive manufacturing, laser processing, etching or any combination of the foregoing. Jaws 10 and 12 may be mass produced in a relatively inexpensive manner and may be suitable for use on disposable devices (e.g., single patient use devices). In some embodiments, jaws 10 and 12 may be produced from a plastic material or a combination of materials, such as metal and plastic, ceramic and plastic, for example. In various embodiments, the mechanical arrangement of jaw assembly 100 may be configured to allow some control over the angle of opening of jaws 10, 12.

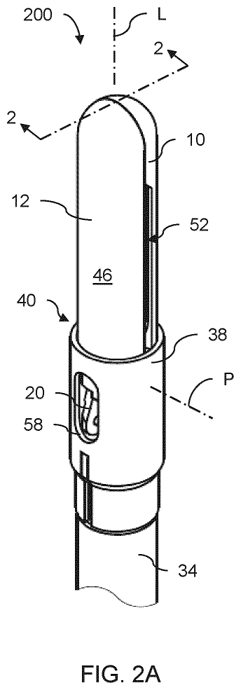

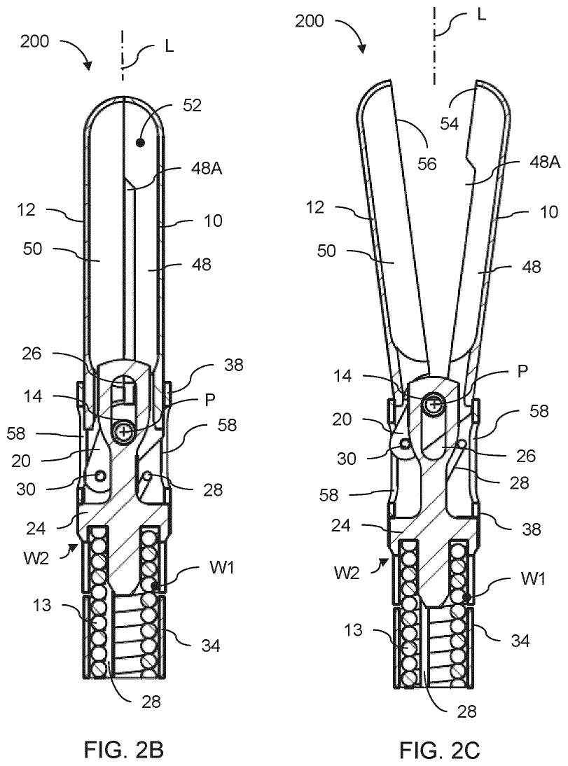

FIGS. 2A-2C illustrate another exemplary embodiment of a biopsy jaw assembly 200 of a biopsy forceps device for harvesting one or more biopsy samples from a patient. Some aspects of jaw assembly 100 described above also apply to jaw assembly 200 and such description is not repeated below. Like reference numerals are used to denote like elements.

In contrast with jaw assembly 100, jaw assembly 200 may comprise first jaw 10 and second jaw 12 that are configured differently from each other to promote the retention of the biopsy samples in only one of jaws 10, 12 during use. Such feature may, for example, cause a row of biopsy samples harvested by jaw assembly 200 to be retained in second jaw 12 as jaws 10, 12 are repeatedly opened and closed and are moved to different locations within the patient to harvest a plurality of biopsy samples.

It is understood that features promoting the retention of the biopsy samples in only one of jaws 10, 12 may be incorporated into any of the jaw assemblies disclosed herein and also into other types of jaw assemblies. In some embodiments, a fenestration of first jaw 10 may be different from a fenestration of second jaw 12 so that, for example, inner surface 50 of second jaw 12 may provide a larger contact area for interfacing with the one or more biopsy samples than inner surface 48 of first jaw 10 to promote adhesion of the one or more biopsy samples to inner surface 50 of second jaw 12 after harvesting instead of inner surface 48 of first jaw 10. In various embodiments, first jaw 10 and second jaw 12 may each have a different number of windows, and/or, first jaw 10 and second jaw 12 may have windows of different sizes. For example, in some embodiments, first jaw 10 may comprise window(s) 48A defined in inner surface 48 of first jaw 10, and, second jaw 12 may be free of windows (e.g., windows 50A in FIGS. 1D and 1G) defined in inner surface 50 of second jaw 12. Accordingly, in some embodiments, face 56 of second jaw 12 may lie substantially entirely the mid-plane that intersects and that is parallel to longitudinal axis L when second jaw 12 is in its closed position. Conversely, a portion of face 54 of first jaw 10 defining window(s) 48A may be offset from the mid-plane when first jaw 10 is in its closed position.

In some embodiments, inner surface 48 and inner surface 50 may cooperatively define sample-retaining volume 52 having a substantially circular or non-circular cross-sectional profile which may be interrupted due to the presence of windows. For example, due to the presence of windows 48A in first jaw 10 and to the lack of windows in second jaw 12, a transverse cross-sectional profile of inner surface 50 of second jaw 12 may have a longer arc length than a corresponding transverse cross-sectional profile of inner surface 48 of first jaw 10.

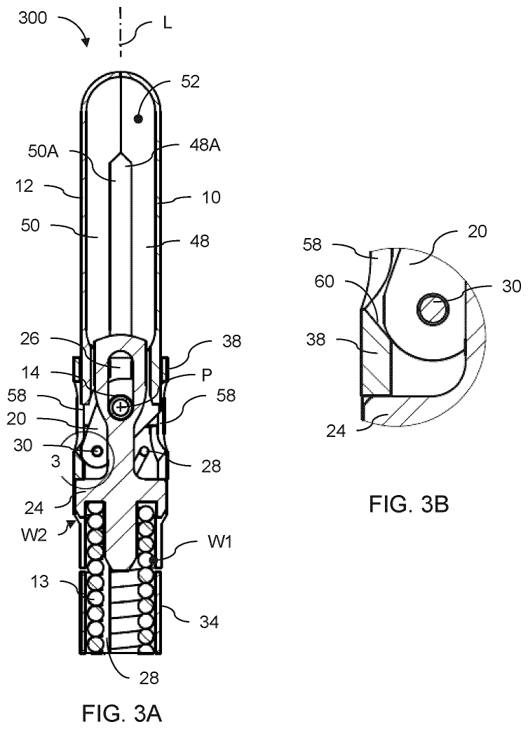

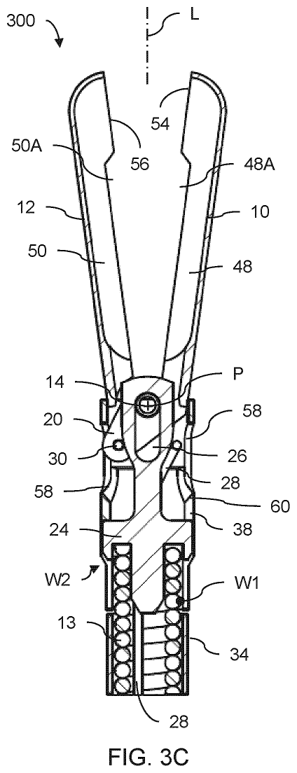

FIGS. 3A-3C illustrate another exemplary embodiment of a biopsy jaw assembly 300 of a biopsy forceps device for harvesting one or more biopsy samples from a patient. Some aspects of the jaw assemblies described above also apply to jaw assembly 300 and such description is not repeated below. Like reference numerals are used to denote like elements.

Jaw assembly 300 may be mostly identical to jaw assembly 100 except for the addition of another guide (e.g., shoulder(s) 60) formed into sleeve 38 for interacting with one or both of arms 20 and 22 of first jaw 10 and second jaw 12 respectively. FIG. 3B illustrates only one shoulder 60 for interacting with arm 20 but it is understood that another, diametrically opposite shoulder 60 may be provided for interacting with arm 22 of second jaw 12. Shoulder(s) 60 may be provided instead of or in addition to urging surface 42 of sleeve 38 for the purpose of enhancing the clamping force of one or both of first jaw 10 and second jaw 12. As jaws 10, 12 are pulled proximally by actuation wires 28 and progressively received into sleeve 38, shoulders 60 may come into contact with arms 20, 22 and urge arms 20, 22 toward longitudinal axis L to thereby supplement the closing moment provided by actuation wires 28 alone. Similarly to the force enhancing effect provided by urging surface 42 of sleeve 38, shoulders 60 may also convert some of the axial force(s) provided by actuation wires 28 into radially-inward forces acting on respective arms 20, 22 and relative to common pivot axis P. This force-enhancement effect may be provided in part by the orientations of shoulders 60 and arms 20, 22 relative to longitudinal axis L. As illustrated by FIGS. 3A and 3B, shoulders 60 may only come into contact with respective arms 20, 22, and thereby enhance the closing moment, at or near the end of the closing manoeuver/stroke.

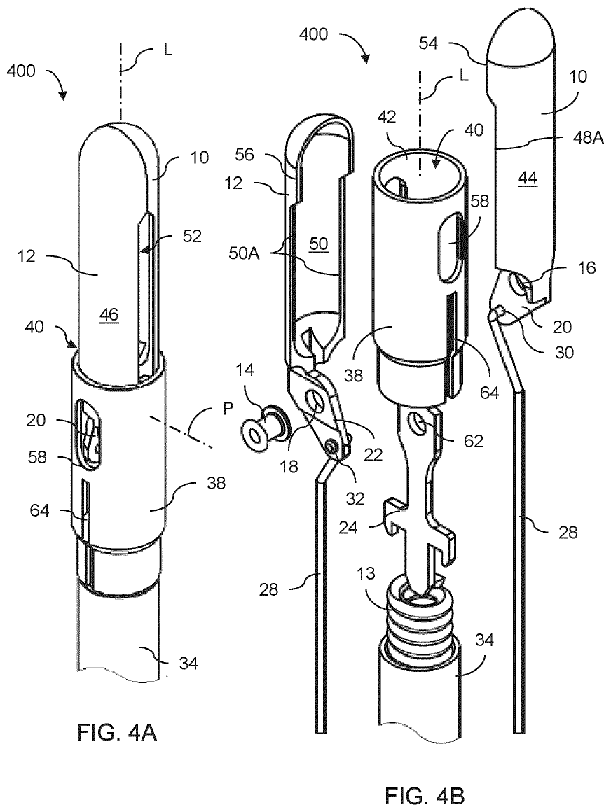

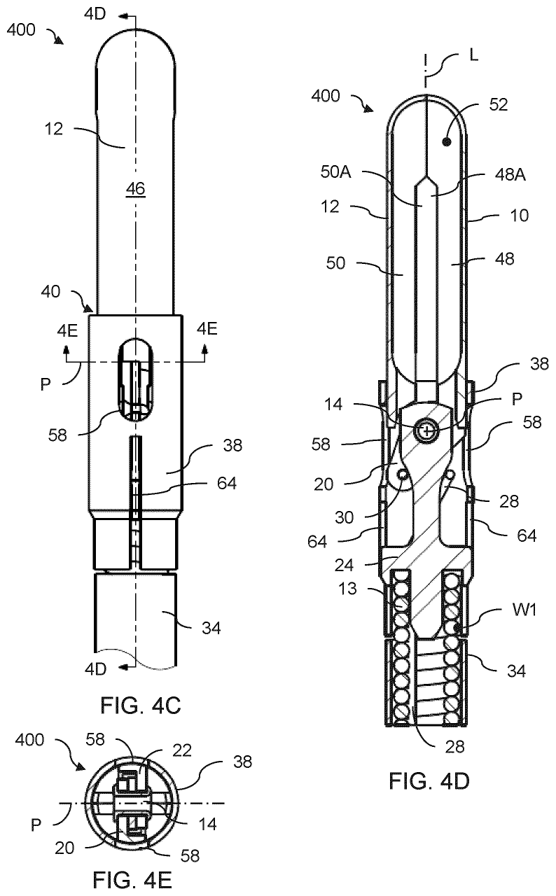

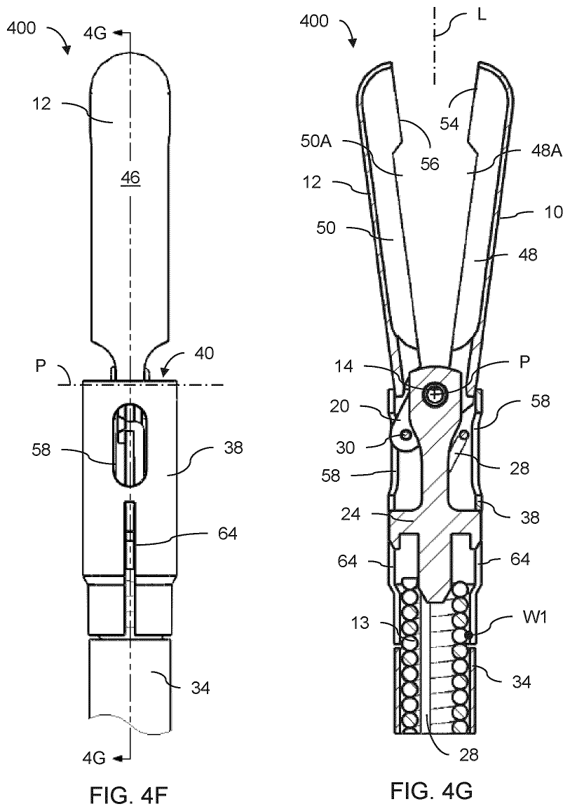

FIGS. 4A-4G illustrate another exemplary embodiment of a biopsy jaw assembly 400 of a biopsy forceps device for harvesting one or more biopsy samples from a patient. Some aspects of the jaw assemblies described above also apply to jaw assembly 400 and such description is not repeated below. Like reference numerals are used to denote like elements.

In contrast with jaw assembly 100, jaw assembly 400 may be configured so that instead of first jaw 10 and second jaw 12 being slidingly engaged with flat member 24, jaws 10, 12 may be pivotally coupled to flat member 24 via hole 62 instead of slot 26 from jaw assembly 100. In order to achieve similar actuation of jaws 10, 12 and clamping force-enhancing effect provided by sleeve 38, flat member 24 may instead be slidingly disposed inside sleeve 38 so that longitudinal movement of flat member 24 to accommodate the actuation of jaws 10, 12 may be guided by slots 64 provided in sleeve 38. In this embodiment, sleeve 38 may be welded to shaft 13 at W1 but flat member 24 may not be welded to shaft 13 in order to permit longitudinal movement of flat member 24 relative to shaft 13.

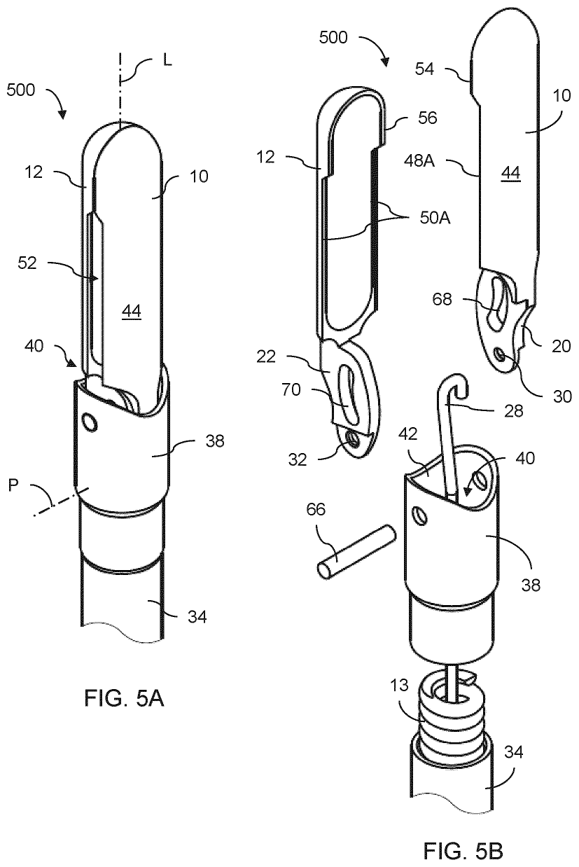

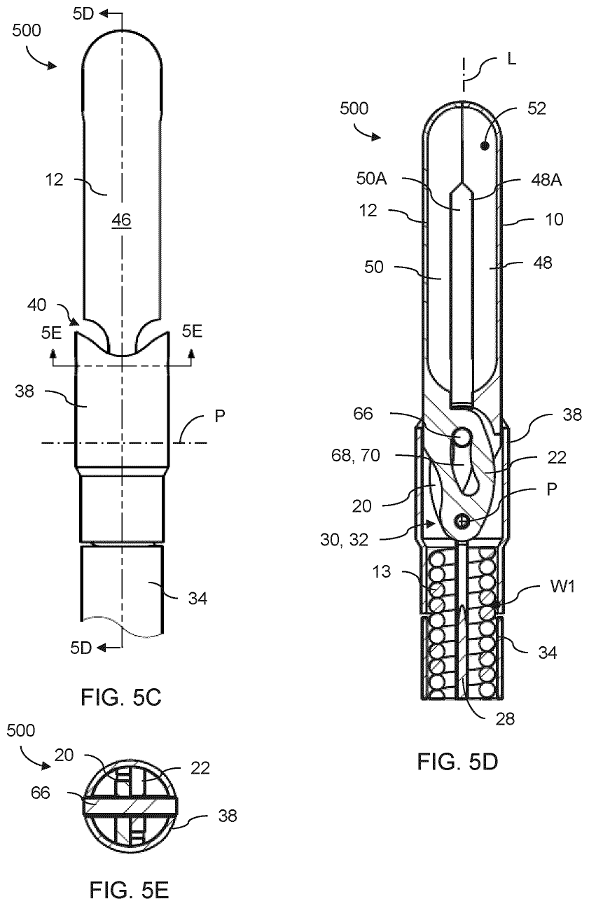

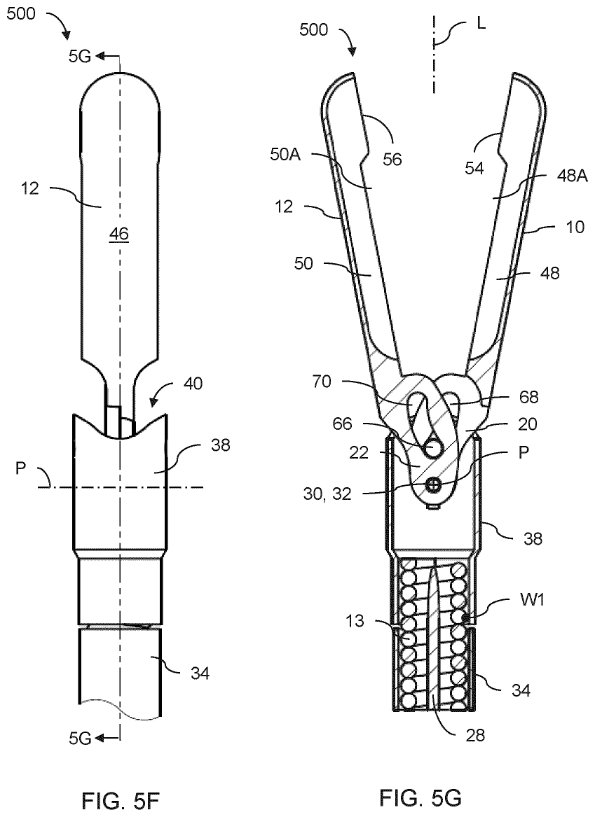

FIGS. 5A-5G illustrate another exemplary embodiment of a biopsy jaw assembly 500 of a biopsy forceps device for harvesting one or more biopsy samples from a patient. Some aspects of the jaw assemblies described above also apply to jaw assembly 500 and such description is not repeated below. For example, assembly 500 may also benefit from the enhanced clamping force of first jaw 10 and second jaw 12 provided by the interaction of sleeve 38 with first jaw 10 and second jaw 12. Like reference numerals are used to denote like elements.

In contrast with the jaw assemblies described above, jaw assembly 500 may be configured so that first jaw 10 and second jaw 12 may be actuated by a single common actuation wire 28 coupled to both arms 20 and 22 at actuation points 30, 32 respectively. In this embodiment, the distal movement of actuation wire 28 may cause both jaws 10, 12 to open and proximal movement of actuation wire 28 may cause both jaws 10, 12 to close. In this embodiment, common pivot axis P may pass through actuation points 30, 32 and the opening/closing movement of jaws 10, 12 may be guided by pin 66, which may be secured to sleeve 38, by way of sliding engagement of pin 66 with slots 68 and 70 formed in arms 20 and 22 respectively as jaws 10, 12 are translated longitudinally by actuation wire 28 during actuation. Pin 66 and corresponding slots 68 and 70 may provide a cam/follower arrangement for controlling the movement of first jaw 10 and second jaw 12.

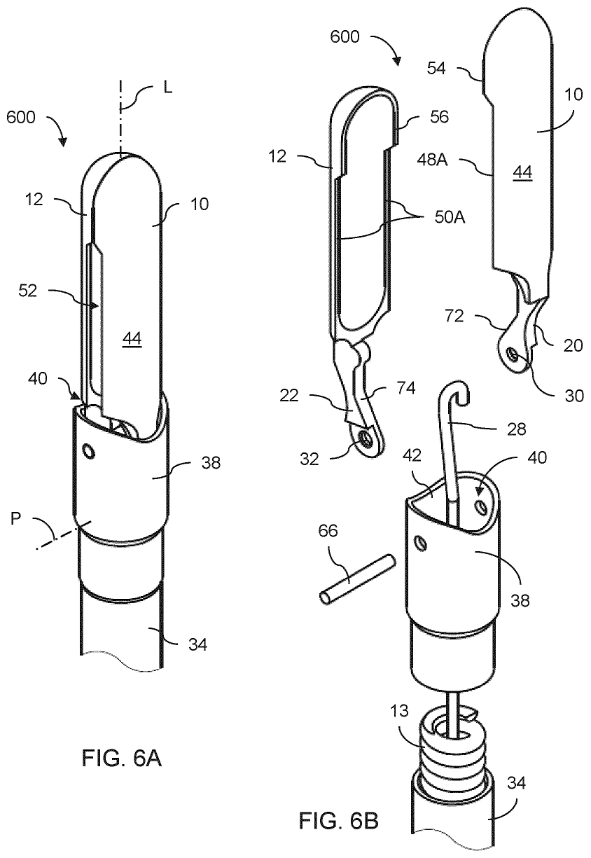

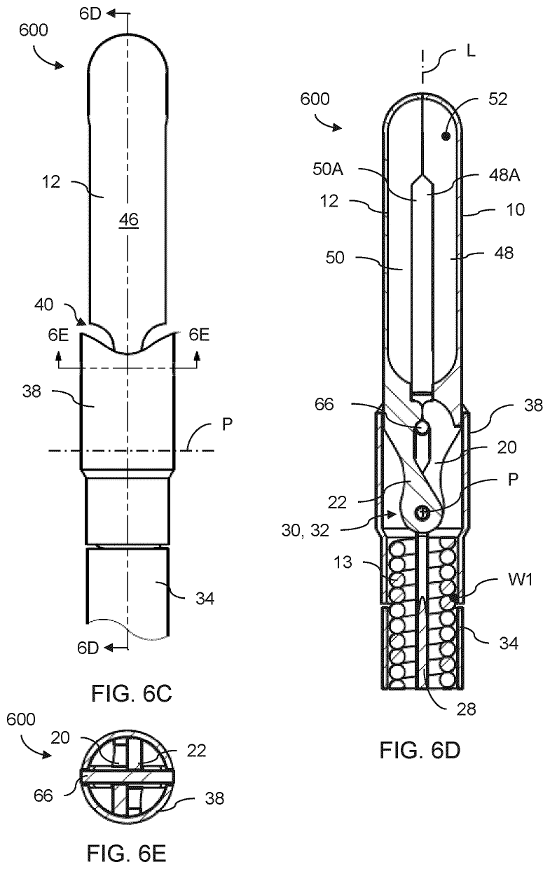

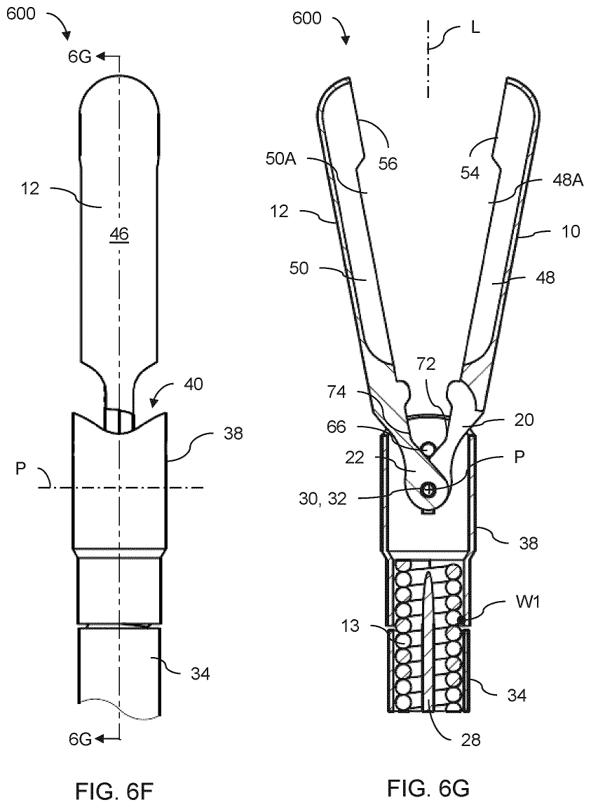

FIGS. 6A-6G illustrate another exemplary embodiment of a biopsy jaw assembly 600 of a biopsy forceps device for harvesting one or more biopsy samples from a patient. Some aspects of the jaw assemblies described above also apply to jaw assembly 600 and such description is not repeated below. For example, assembly 600 may also benefit from the enhanced clamping force of first jaw 10 and second jaw 12 provided by the interaction of sleeve 38 with first jaw 10 and second jaw 12. Like reference numerals are used to denote like elements.

Like jaw assembly 500, jaw assembly 600 may also be configured so that first jaw 10 and second jaw 12 may be actuated by a single common actuation wire 28 coupled to both arms 20 and 22 at actuation points 30, 32 respectively. In this embodiment, the opening/closing movement of jaws 10, 12 may be guided by pin 66, which may be secured to sleeve 38, by way of sliding engagement of pin 66 with surfaces 72 and 74 formed on arms 20 and 22 respectively as jaws 10, 12 are translated longitudinally by actuation wire 28 during actuation. Pin 66 and corresponding surfaces 68 and 70 may provide a cam/follower arrangement for controlling the movement of first jaw 10 and second jaw 12.

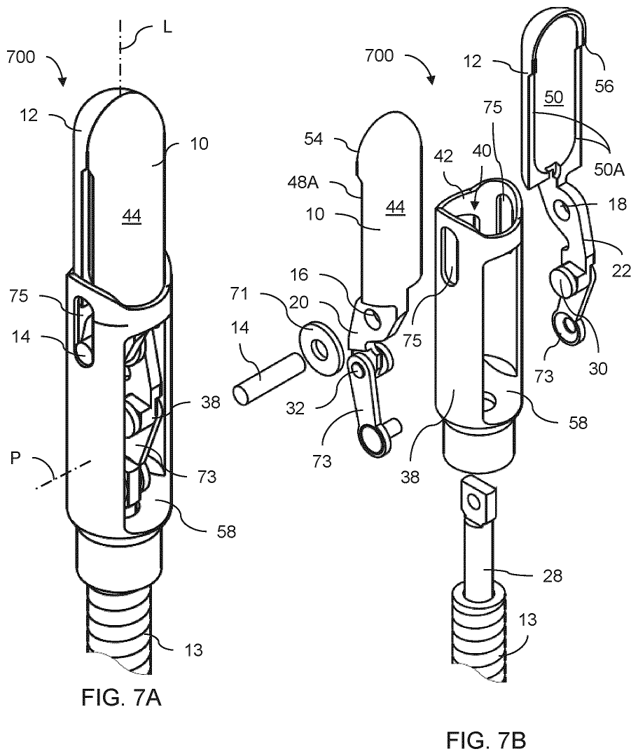

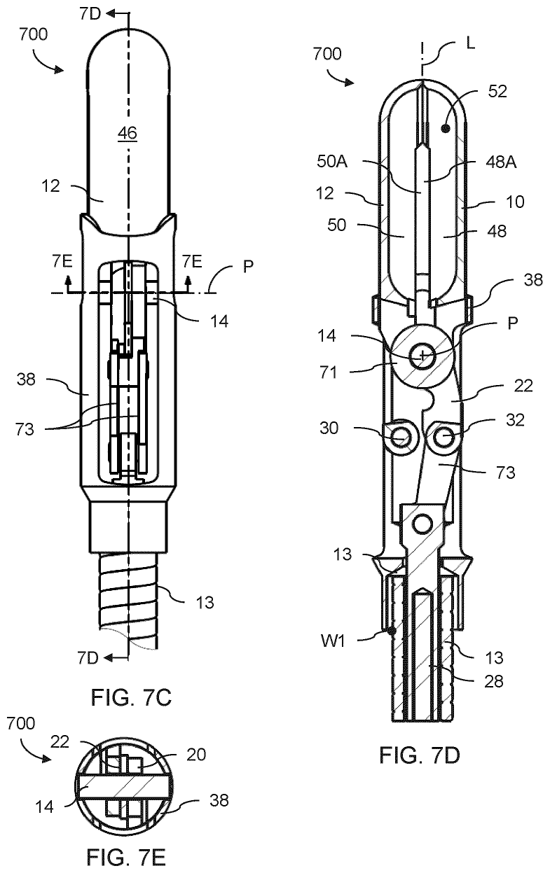

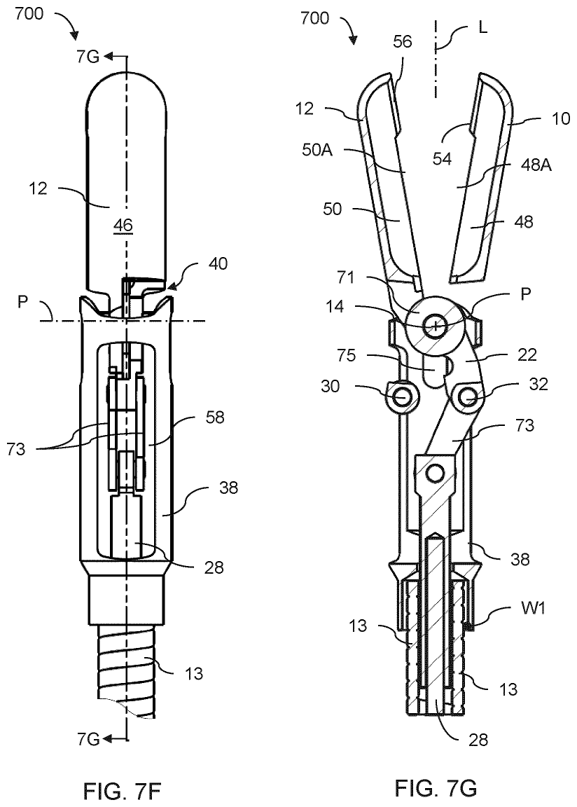

FIGS. 7A-7G illustrate another exemplary embodiment of a biopsy jaw assembly 700 of a biopsy forceps device for harvesting one or more biopsy samples from a patient. Some aspects of the jaw assemblies described above also apply to jaw assembly 700 and such description is not repeated below. For example, assembly 700 may also benefit from the enhanced clamping force of first jaw 10 and second jaw 12 provided by the interaction of sleeve 38 with first jaw 10 and second jaw 12. Like reference numerals are used to denote like elements.

In contrast with jaw assembly 100, jaw assembly 700 may be configured so that instead of first jaw 10 and second jaw 12 being slidingly engaged with flat member 24 (see FIG. 1B), jaws 10, 12 may be slidingly engaged with sleeve 38 via pivot member 14 and slots 75 formed in sleeve 38 or other suitable means. For example, first jaw 10 and second jaw 12 may be pivotally coupled to common pivot member 14 and opposite ends of pivot member 14 may be engaged with diametrically opposed slots 75 so that common pivot member 14 may be translatable relative to sleeve 38 when jaws 10, 12 are actuated via actuation wire(s) 28. Common pivot member 14 may extend through washer 71, which may be disposed between arms 20, 22 of first and second jaws 10, 12. In various embodiments, slots 75 may extend partially or completely through the wall of sleeve 38. In the case where slots 75 extend completely through the wall of sleeve 38, common pivot member 14 may be retained in position by a suitable retaining clip (not shown) or by press-fitting with one of arms 20, 22 of jaws 10, 12, or by press-fitting with washer 71 for example. In various embodiments, slots 75 may be formed on a radially-inner surface of sleeve 38 and may define one or more grooves, tracks and/or rails for engagement with common pivot member 14 for guiding the translation movement of common pivot member 14 along longitudinal axis L relative to sleeve 38. In various embodiments, slots 75 may each comprise a distal end or other suitable means of stopping the distal translation movement of common pivot member 14 when jaws 10, 12 are opened and common pivot member 14 is consequently moved distally during such opening. Such distal ends of slots 75 may define the maximum distal position of jaws 10, 12 relative to sleeve 38 when jaws 10, 12 are fully open. In some embodiments, jaw assembly 700 may comprise a single actuation wire 28 that is operatively coupled to both arms 20, 22 of jaws 10, 12 via respective links 73.

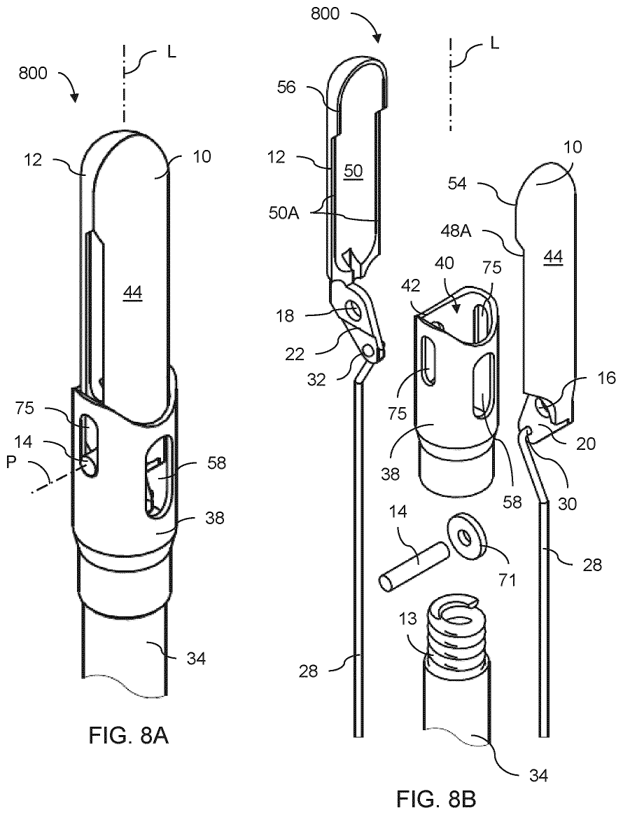

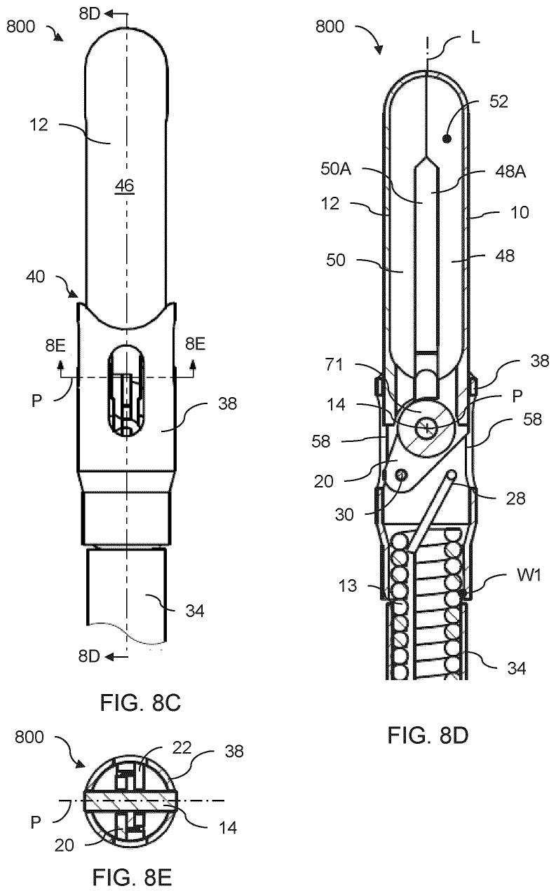

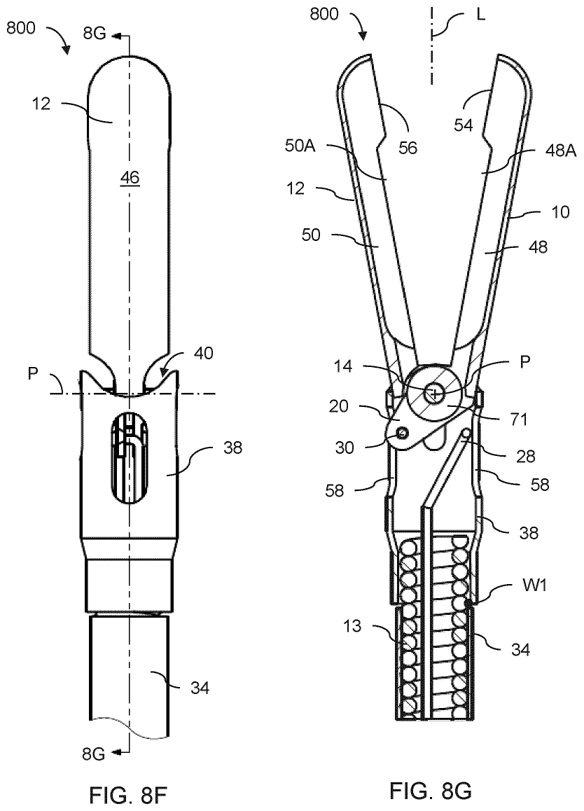

FIGS. 8A-8G illustrate another exemplary embodiment of a biopsy jaw assembly 800 of a biopsy forceps device for harvesting one or more biopsy samples from a patient. Some aspects of the jaw assemblies described above also apply to jaw assembly 800 and such description is not repeated below. For example, assembly 800 may also benefit from the enhanced clamping force of first jaw 10 and second jaw 12 provided by the interaction of sleeve 38 with first jaw 10 and second jaw 12. Like reference numerals are used to denote like elements.

Similar to jaw assembly 700, jaw assembly 800 may also comprise diametrically opposed slots 75 formed in sleeve 38 for sliding engagement with opposite ends of common pivot member 14. Instead of a single actuation wire 28, jaw assembly 800 may comprise separate actuation wires 28 respectively associated with first jaw 10 and second jaw 12. Actuation wires 28 may be actuatable in unison to cause opening and closing of jaws 10 and 12. In this embodiment, jaws 10 and 12 may be pivotally coupled to common pivot member 14 via holes 16 and 18 respectively. In some embodiments, common pivot member 14 may be retained in a manner described above in relation to jaw assembly 700.

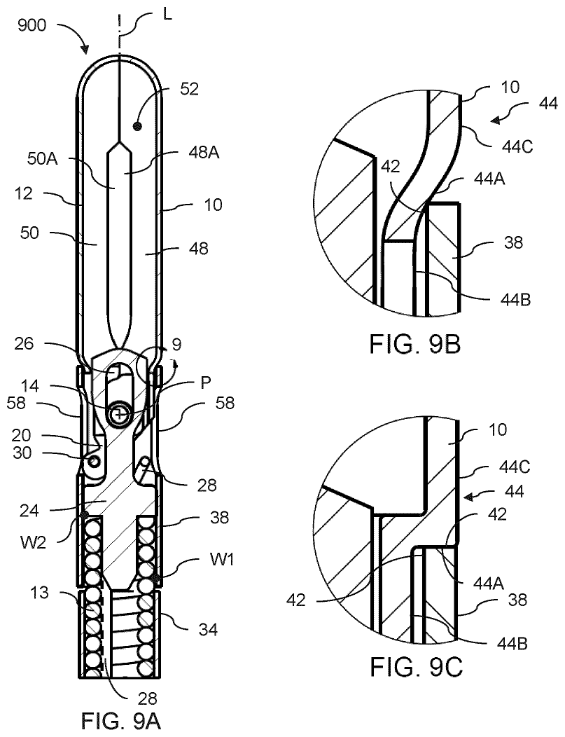

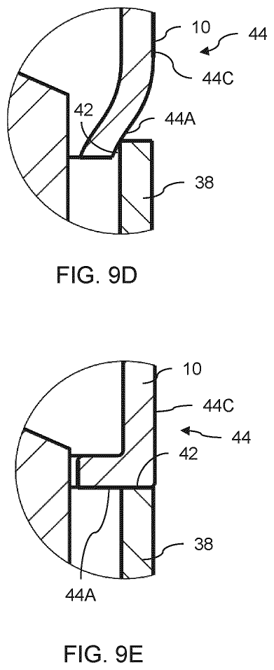

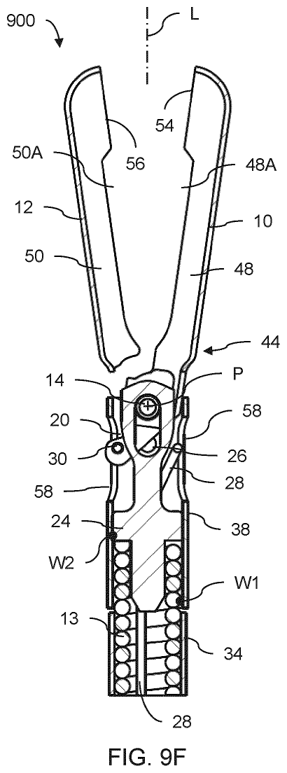

FIGS. 9A-9F illustrate another exemplary embodiment of a biopsy jaw assembly 900 of a biopsy forceps device for harvesting one or more biopsy samples from a patient. Some aspects of the jaw assemblies described above also apply to jaw assembly 900 and such description is not repeated below. Like reference numerals are used to denote like elements.

The actuation mechanism of jaw assembly 900 is similar to that of jaw assembly 100 described above. However, in contrast with jaw assembly 100, jaw assembly 900 may have a sleeve 38 having a similar or a smaller outer diameter than that of jaw assembly 100 to promote size reduction of (i.e., a slimmer) jaw assembly. For example, in some embodiments, the outer diameter of sleeve 38 may be substantially the same as or less than that defined by jaws 10 and 12 when in their respective closed positions. In particular, sleeve 38 may define a maximum radially-outer dimension that is the same or less than a maximum radially-outer dimension cooperatively defined by first jaw 10 and second jaw 12 when first jaw 10 and second jaw 12 are closed.

Flat member 24 may be secured to sleeve 38 by (e.g., lap) welding at W2 or by other suitable means. In some embodiments, jaws 10 and 12 may be pivotally mounted to flat member 24 via the engagement of common pivot member 14 with slot 26. An assembly comprising jaws 10 and 12 and flat member 24 may be inserted into sleeve 38 as unit prior to welding or otherwise securing flat member 24 to sleeve 38. Sleeve 38 may be secured to shaft 13 by (e.g., lap) welding at W1 or by other suitable means, such as crimping, fastening, soldering, brazing, gluing or press fitting for example.

In some embodiments, in order to permit translation of jaws 10 and 12 into and out of sleeve 38 along longitudinal axis L, jaws 10 and 12 may comprise a proximal portion 44B of reduced outer diameter that is configured to be received in aperture 40 of sleeve 38. In reference to FIGS. 9B and 9C, first jaw 10 may comprise a radially-outer surface 44 having transition surface portion 44A extending between two regions of the radially-outer surface 44 having different radially-outer dimensions relative to the longitudinal axis L. For example, transition surface portion 44A may provide a radial transition between a radially-smaller proximal portion 44B of first jaw 10 and a radially-larger distal portion 44C of first jaw 10. It is understood that second jaw 12 may also be configured as shown in FIGS. 9B and 9C and comprise its own transition surface portion.

FIG. 9B illustrates one exemplary embodiment of transition surface portion 44A where transition surface portion 44A is oblique to longitudinal axis L. FIG. 9C illustrated another exemplary embodiment of transition surface portion 44A where transition surface portion 44A is substantially transverse to longitudinal axis L. During closing of jaws 10, 12, urging surface(s) 42 of sleeve 38 may engage with transition surface portion 44A to supplement the clamping force applied by jaws 10, 12 near or at the end of the closing movement of jaws 10, 12.

In some embodiments, transition surface portion 44A may not necessarily be between two regions of the radially-outer surface 44 having different radially-outer dimensions relative to the longitudinal axis L. FIGS. 9C and 9D each illustrate an embodiment where transition surface portion 44A extends radially inwardly from radially-larger distal portion 44C of jaw 10 but that is not necessarily connected to radially-smaller proximal portion 44B. Accordingly, radially-smaller proximal portion 44B of jaw 10 may not be necessary to supplement the clamping force.

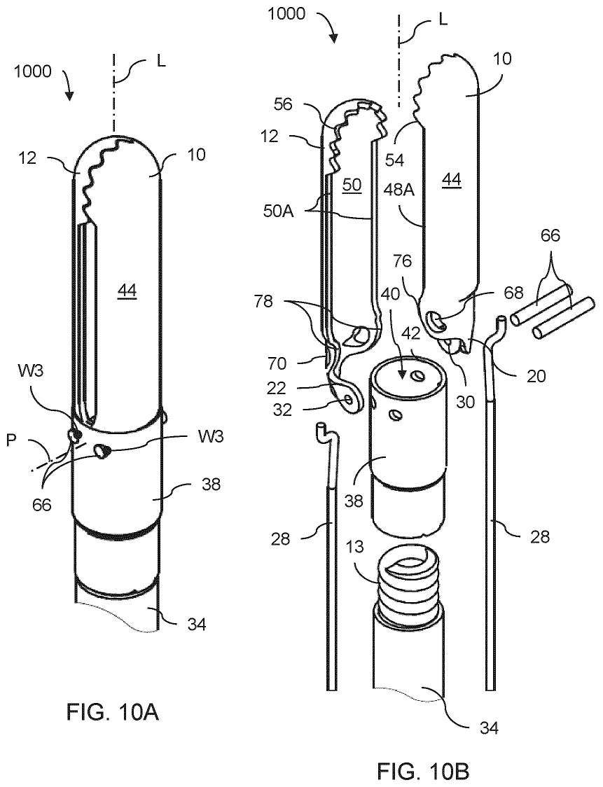

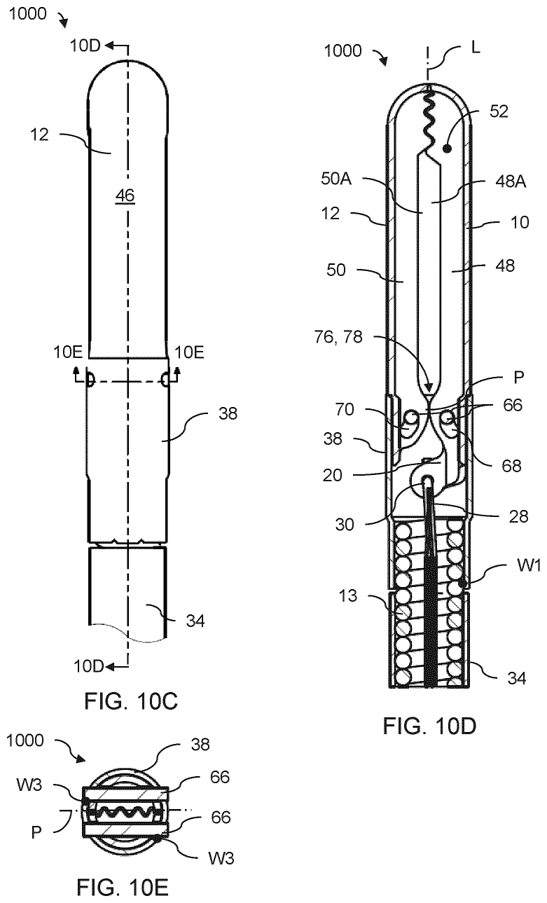

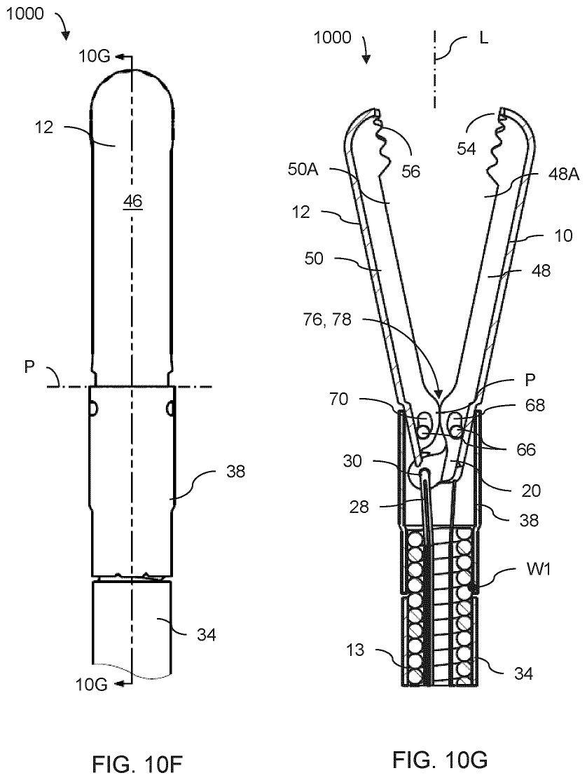

FIGS. 10A-10G illustrate another exemplary embodiment of a biopsy jaw assembly 1000 of a biopsy forceps device for harvesting one or more biopsy samples from a patient. Some aspects of the jaw assemblies described above also apply to jaw assembly 1000 and such description is not repeated below. For example, assembly 1000 may also benefit from the enhanced clamping force of first jaw 10 and second jaw 12 provided by the interaction of sleeve 38 with first jaw 10 and second jaw 12. Like reference numerals are used to denote like elements. It is understood that in various embodiments of biopsy jaw assemblies disclosed herein, one or both faces 54, 56 of jaws 10, 12 may be serrated as illustrated in FIG. 10A-10G for example.

Jaw assembly 1000 may comprise a separate guide pin 66 associated with each of jaws 10, 12 and secured to sleeve 38. Each guide pin 66 may guide the opening/closing movement of a respective jaw 10, 12 by way of sliding engagement of pins 66 with respective slots 68 and 70 formed in jaws 10, 12 respectively as jaws 10, 12 are translated longitudinally by respective actuation wires 28 during actuation. Guide pins 66 may be configured to limit the movement of jaws 10, 12 along longitudinal axis L during the actuation of jaws 10, 12. Guide pins 66 may be substantially parallel when opposite ends of pins 66 are engaged with sleeve 38. Guide pins 66 and corresponding slots 68 and 70 may provide a cam/follower arrangement for controlling the movement of first jaw 10 and second jaw 12. As illustrated in FIGS. 10D and 10G, the position of common pivot axis P may vary (e.g., along longitudinal axis L) relative to sleeve 38 as jaws 10, 12 are actuated.

In some embodiments, first jaw 10 of jaw assembly 1000 may comprise one or more rocking surfaces 76 and second jaw 12 may comprise one or more corresponding rocking surfaces 78. During the pivotal movement of jaws 10, 12, rocking surface(s) 76 may interface with rocking surface(s) 78 and guide the movement of jaws 10, 12. Rocking surfaces 76, 78 may be arcuate and convex when viewed from the orientation of FIGS. 10D and 10G. Common pivot axis P may be disposed at a contact interface between rocking surfaces 76, 78. As the contact interface (e.g., two contact points) between the opposed rocking surfaces 76, 78 moves during actuation of jaws 10, 12, the common pivot axis P also moves along longitudinal axis L as shown in FIGS, 10D and 10G.

The opposite ends of guide pins 66 may be secured to sleeve 38 by (e.g., laser) welding at locations W3 for example (see FIG. 10A). In some embodiments, the arrangement of jaw assembly 1000 may reduce or eliminate the need for relatively large openings in sleeve 38 such as slots 58 and 75 as shown in previous embodiments. The absence of such openings in sleeve 38 may eliminate the possibility of wires protruding out of sleeve 38 via such openings and consequently promote the safety of the patient and operator also reduce the risk of damaging the endoscope.

In some embodiments, the arrangement of jaw assembly 1000 may facilitate a relatively short proximal movement of jaws 10, 12 during the closing of jaws 10, 12 to promote a good harvesting performance of jaw assembly 1000. The arrangement of jaw assembly 1000 may also facilitate manufacturing by press working in some embodiments by reducing the severity of bends in the parts relative to some other embodiments.

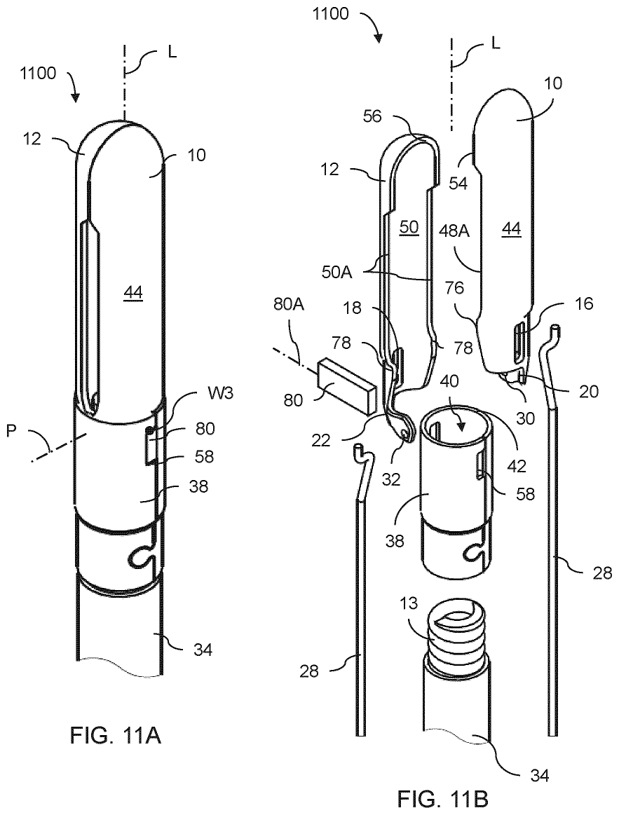

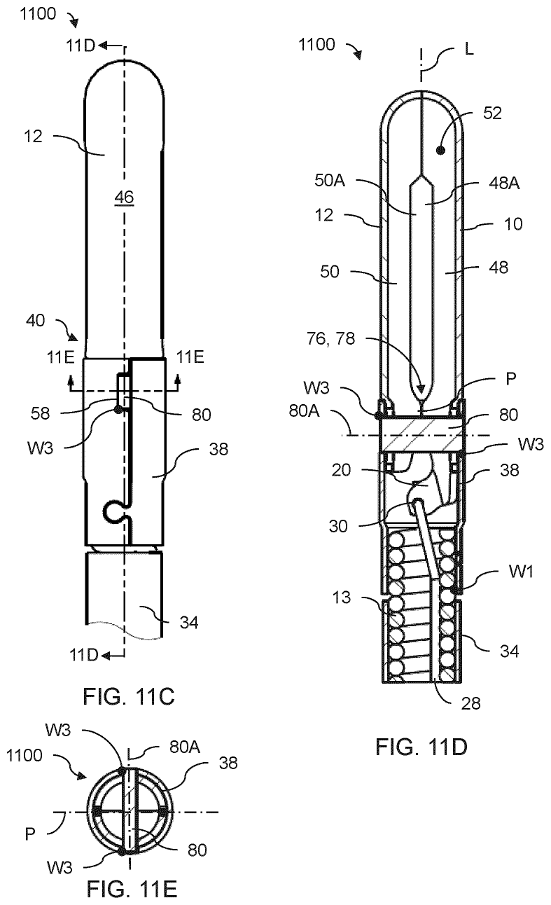

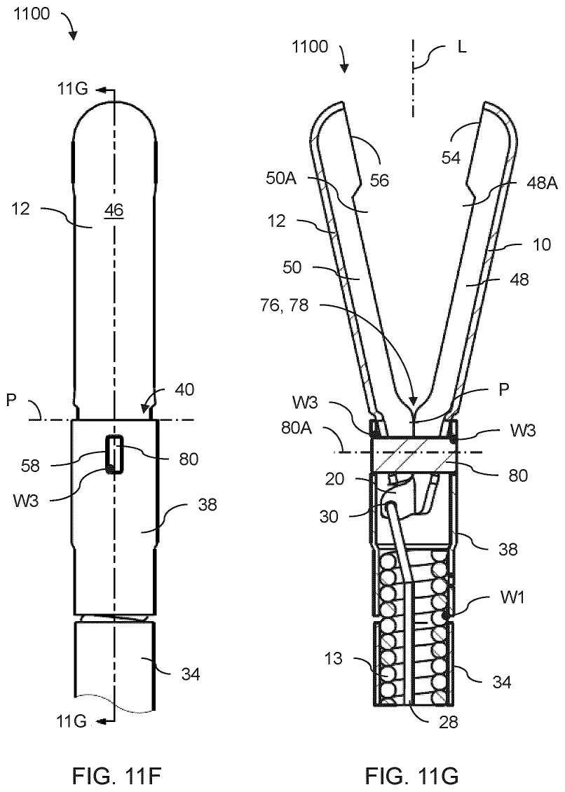

FIGS. 11A-11G illustrate another exemplary embodiment of a biopsy jaw assembly 1100 of a biopsy forceps device for harvesting one or more biopsy samples from a patient. Some aspects of the jaw assemblies described above also apply to jaw assembly 1100 and such description is not repeated below. For example, assembly 1100 may also benefit from the enhanced clamping force of first jaw 10 and second jaw 12 provided by the interaction of sleeve 38 with first jaw 10 and second jaw 12. Like reference numerals are used to denote like elements.

Jaw assembly 1100 may comprise a single common guide pin 80 to which first jaw 10 and second jaw 12 may be slidingly engaged via respective holes 16 and 18. Common guide pin 80 may be oriented generally in a plane of the movement of jaws 10, 12 during actuation of jaws 10, 12. For example, a longitudinal axis 80A of common guide pin 80 may be transverse to common pivot axis P.

The ends of guide pin 80 may be secured to sleeve 38 by engagement with slots 58 and by (e.g., laser) welding at locations W3 for example (see FIG. 11A). In some embodiments, the arrangement of jaw assembly 1100 may also reduce or eliminate the need for relatively large openings in sleeve 38 as shown in previous embodiments. The absence of such openings in sleeve 38 may eliminate the possibility of wires protruding out of sleeve 38 via such openings and consequently promote the safety of the patient and operator also reduce the risk of damaging the endoscope.

Common guide pin 80 may be configured to limit the movement of jaws 10, 12 along longitudinal axis L or the central axis of sleeve 38 during the actuation of jaws 10, 12. Common guide pin 80 may be configured to stabilize and promote alignment of jaws 10, 12 during actuation. Common guide pin 80 may comprise a rectangular bar. For example, common guide pin 80 may have a rectangular cross-sectional profile transverse to its longitudinal axis 80A. A dimension of the transverse cross-sectional profile along the longitudinal axis L may be greater than a dimension of the transverse cross-sectional profile transverse to the longitudinal axis L.

Similar to jaw assembly 1000, jaw 10 of jaw assembly 1100 may comprise one or more rocking surfaces 76 and jaw 12 may comprise one or more rocking surfaces 78. During the pivotal movement of jaws 10, 12, rocking surface(s) 76 may interface with rocking surface(s) 78 and guide the movement of jaws 10, 12. Common pivot axis P may be disposed at a contact interface between rocking surfaces 76, 78. As the contact interface (e.g., two contact points) between the opposed rocking surfaces 76, 78 moves during actuation of jaws 10, 12, the common pivot axis P also moves along longitudinal axis L as shown in FIGS, 11 D and 11G.

The arrangement of jaw assembly 1100 may also facilitate manufacturing by having fewer parts compared to jaw assembly 1000 for example. The arrangement of jaw assembly 1100 may also facilitate assembly by eliminating the need for riveting or eyeleting of a main pivot pin.

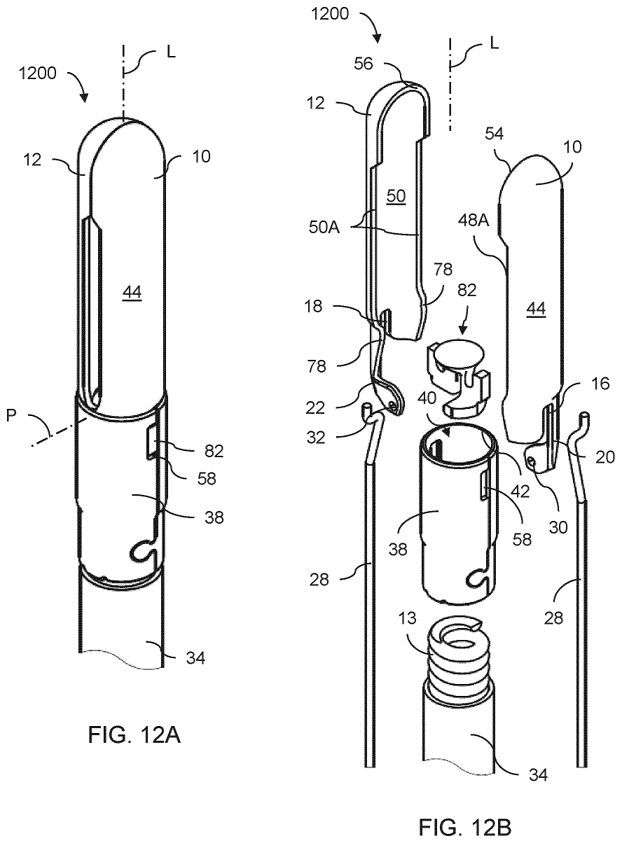

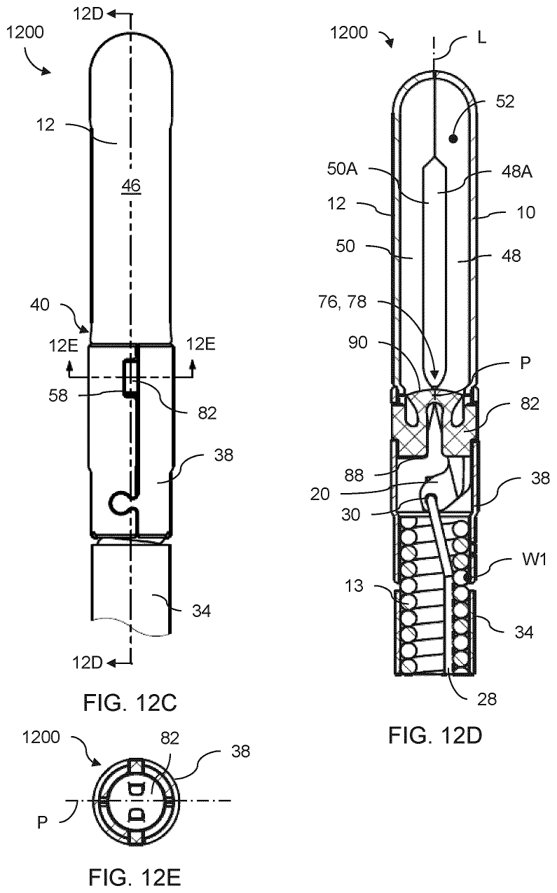

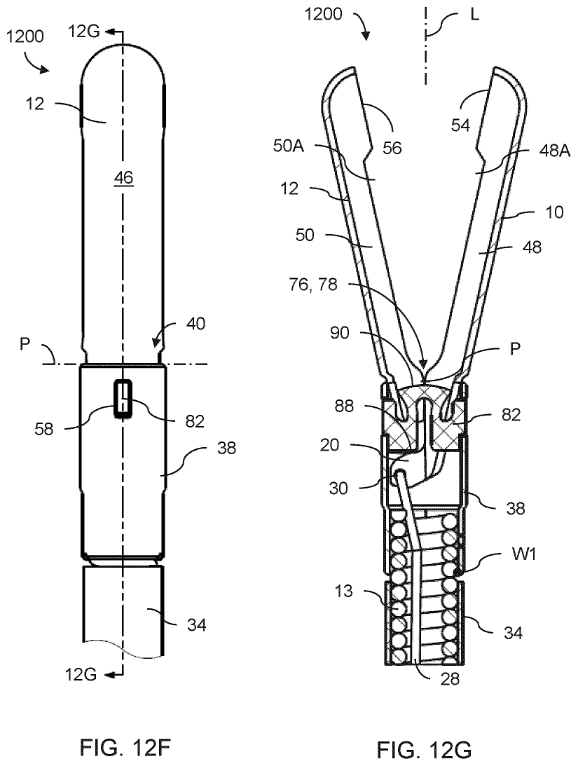

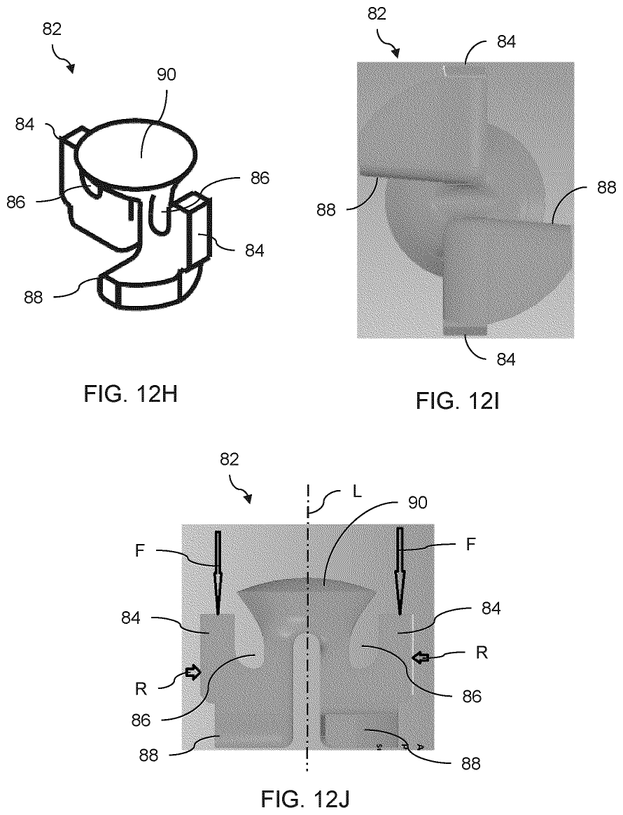

FIGS. 12A-12G illustrate another exemplary embodiment of a biopsy jaw assembly 1200 of a biopsy forceps device for harvesting one or more biopsy samples from a patient. FIGS. 12H-12J illustrate guide insert 82 which may be part of jaw assembly 1200. Some aspects of the jaw assemblies described above also apply to jaw assembly 1200 and such description is not repeated below. For example, assembly 1200 may also benefit from the enhanced clamping force of first jaw 10 and second jaw 12 provided by the interaction of sleeve 38 with first jaw 10 and second jaw 12. Like reference numerals are used to denote like elements.

Jaw assembly 1200 may comprise guide insert 82 to which first jaw 10 and second jaw 12 may be slidingly engaged via respective holes 16 and 18. Guide insert 82 may function similarly to common guide pin 80 of jaw assembly 1100 as described above with respect to guiding the movement of the first and second jaws 10, 12 during actuation. In some embodiments, guide insert 82 may be molded from a suitable plastic material. In some embodiments, guide insert 82 may be integrally formed (e.g., molded as one piece) to have a unitary construction. Guide insert 82 may be resiliently inserted into sleeve 38 for ease of assembly and secured to sleeve 38 via engagement with one or more sleeve features. For example, in reference to FIGS. 12H-12J, guide insert 82 may comprise one or more resilient features 84 for engagement with one or more corresponding slots 58 (see FIG. 12B) or other suitable receptacles formed in sleeve 38. Common pivot axis P may be disposed at a contact interface between rocking surfaces 76, 78.

In reference to FIG. 12J, resilient features 84 may be disposed in a radially-opposed relationship relative to longitudinal axis L or the central axis of sleeve 38. Resilient features 84 may comprise tabs that are resiliently movable in a radially inward direction relative to the longitudinal axis L upon the application of a radially-inward force (see arrows R) on the respective resilient feature 84. Guide insert 82 may comprise one or more cutouts 86 to facilitate resilient and radially inward movement of the one or more resilient features 84. Guide insert 82 may comprise one or more stoppers 88 for limiting an opening movement of first jaw 10 and/or second jaw 12. Guide insert 82 may comprise surface 90 defining a proximal end of sample-retaining volume 52 (see FIG. 12D) cooperatively defined by first and second jaws 10, 12.

The configuration of guide insert 82 may facilitate the assembly of jaw assembly 1200 by facilitating the insertion of guide insert 82 into sleeve 38 via aperture 40 (see FIG. 12B) and also facilitating the securing of guide insert 82 with sleeve 38 by the engagement of resilient features 84 with respective slots 58. For example, an outer diametric dimension of guide insert 82 at the positions of resilient features 84 may initially be slightly greater than an inner diameter of sleeve 38 so that the insertion of guide insert 82 into sleeve 38 may force resilient features 84 to resiliently deflect radially inwardly as resilient features frictionally engage and slide against an inside surface of sleeve 38. Guide insert 82 may be inserted into sleeve 38 via aperture 40 and pushed in the proximal direction along longitudinal axis L (e.g., see arrows F in FIG. 12J). As each resilient feature 84 becomes aligned with its respective slot 58 of corresponding shape, resilient features 84 move radially outwardly back toward their original non-deflected shapes/positions so as to occupy and become engaged with slots 58 and thereby secure or lock guide insert 82 into sleeve 38. In other words, each resilient feature 84 may spring back toward its original non-deflected shape and click into a respective slot 58 formed in sleeve 38 to secure guide insert 82 with sleeve 38 by providing a positive locking arrangement. The use of guide insert 82 may facilitate assembly of jaw assembly 1200 also by not requiring welding of guide insert 82 with sleeve 38.

Stoppers 88 of guide insert 82 may serve to limit an opening movement of one or more of first and second jaws 10, 12. As illustrated in FIG. 12G, each stopper 88 may be configured to engage a respective arm 20, 22 of first and second jaws 10, 12 so as to serve as a hard stop for limiting the opening movement of its respective first and second jaw 10, 12 during actuation of jaw assembly 1200. In other words, each stopper 88 may serve to interfere with the movement of a respective arm 20, 22 of first and second jaws 10, 12.

Surface 90 of guide insert 82 may define a proximal end of sample-retaining volume 52 cooperatively defined by first and second jaws 10, 12 as shown in FIG. 12D. Surface 90 may prevent consecutively collected biopsy samples from moving too far proximally relative to first and second jaws 10, 12 and potentially get caught or interfere with moving components of jaw assembly 1200. In some embodiments, surface 90 may have a generally circular shape when viewed along longitudinal axis L. In various embodiments, guide insert 82 may comprise a spike, a fork, a pusher or other means for engagement with one or more biopsy samples in addition to or instead of surface 90.

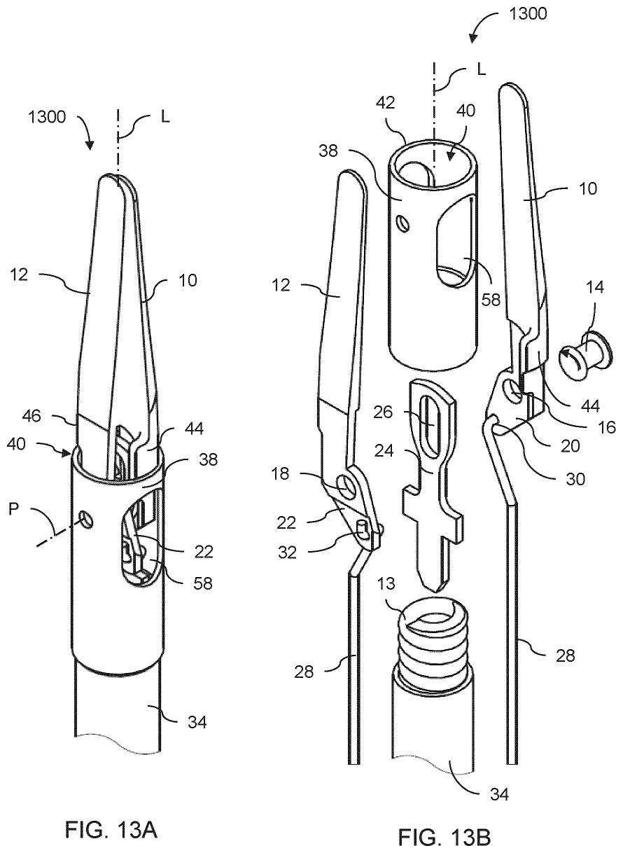

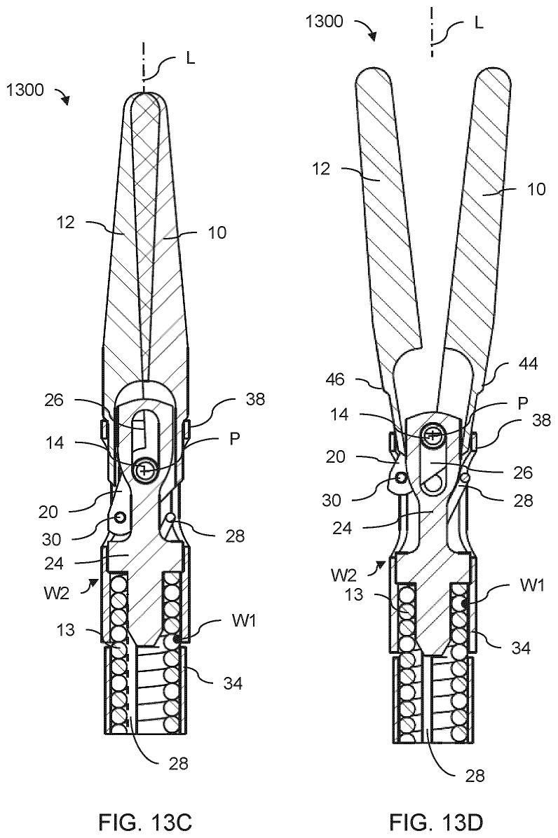

FIGS. 13A-13D illustrate an exemplary embodiment of an end effector jaw assembly 1300 for an endoscopic surgical tool. Some aspects of jaw assemblies described above also apply to jaw assembly 1300 and such description is not repeated below. In the exemplary embodiment shown, the actuation mechanism of jaw assembly 1300 is generally similar to that of jaw assembly 100 described above. Like reference numerals are used to denote like elements.

Instead of jaws 10, 12 configured to harvest biopsy samples as described above, first and second jaws 10, 12 of jaw assembly 1300 are cooperating scissor blades for the purpose of illustrating that aspects of jaw assemblies disclosed herein are not limited to biopsy forceps devices. For example, the enhanced clamping force and other benefit(s) provided by embodiments of jaw assemblies disclosed herein can also be applicable to other types of instruments such as surgical and/or endoscopic tools. As explained above, aspects of this disclosure can be applicable to other tools such as surgical clip appliers, scissors, grabbers, retrievers, suturing devices, punches and any combination of these tools.

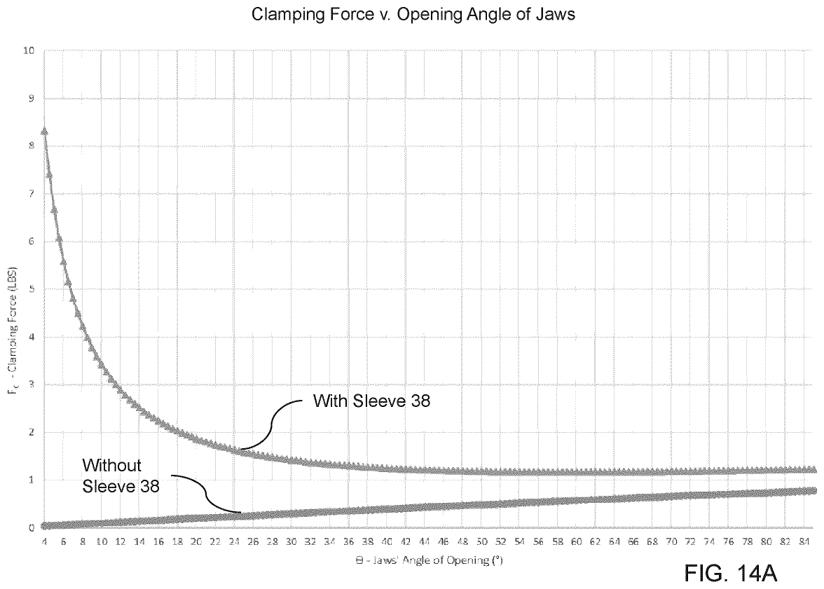

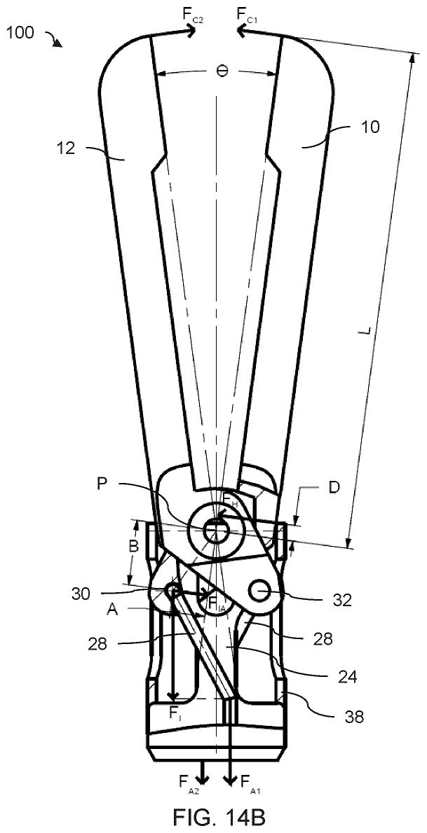

FIG. 14A shows an exemplary graph of the clamping force F_{C} versus the angle θ of opening of first and second jaws 10, 12 to illustrate the effect of sleeve 38 on the clamping force F_{C} as a function of angle θ of opening and FIG. 14B shows an enlarged representation of jaw assembly 100 with part of sleeve 38 cut away to expose the interior thereof. As explained above, the interaction of sleeve 38 with first jaw 10 and second jaw 12 may supplement the closing moment of first jaw 10 and of second jaw 12 provided via the forces applied by actuation wires 28 at the respective actuation points 30 and 32 offset from common pivot axis P. FIG. 14A illustrates the difference in clamping force F_{c} obtained by way of the actuation wires 28 alone (i.e., without sleeve 38) and obtained using sleeve 38 to supplement the clamping force from the actuation wires 28 alone. FIG. 14A shows that as jaws 10, 12 approach their closed configuration (i.e., lower angle of opening), sleeve 38 provides a significant increase in clamping force. It is understood that sleeve 38 may supplement the clamping force in other embodiments of jaw assemblies disclosed herein.

In reference to the nomenclature in FIG. 14B, the clamping force F_{C} may be represented by F_{C}=F_{C1}+F₂ where F_{C} is the total clamping force, F_{C1} is the clamping force of first jaw 10 and F₂ is the clamping force of second jaw 12. The force applied to actuation wires 28 may be represented as F_{A}=F_{A1}+F_{A2} wherein F_{A} is the total force applied to actuation wires 28 for closing jaws 10, 12, F_{A1}, is the force applied to actuation wire 28 coupled to jaw 10, and F_{A2} is the force applied to actuation wire 28 coupled to jaw 12. The angle of opening of jaws 10, 12 is represented by θ. The graph of FIG. 14A, illustrates the clamping force F_{C} as a function of angle θ of opening for an exemplary jaw assembly 100 with the following parameters: dimension A=70 mm, dimension B=1.30 mm, dimension D=0.317 mm, dimension L=10 mm and F_{A1}=F_{A2}=5 lbs (22.2 N).

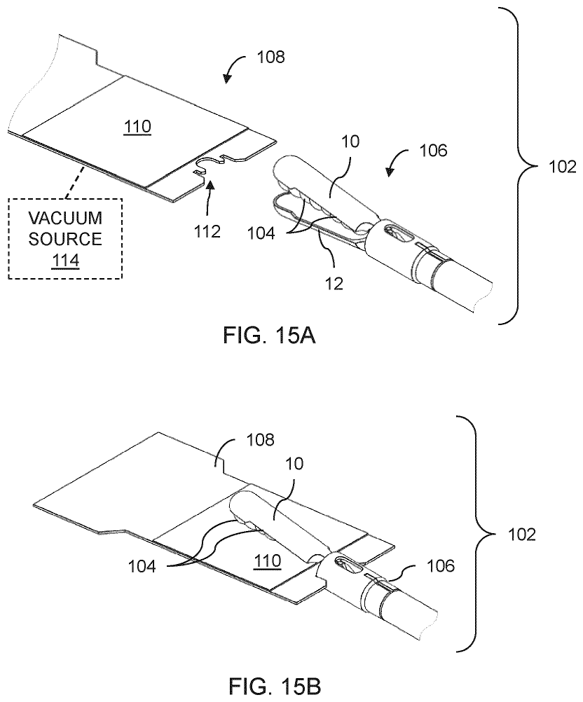

FIGS. 15A and 15B show perspective views of kit 102 for harvesting one or more biopsy samples 104. Kit 102 may comprise biopsy forceps device 106 and collection member 108. Biopsy forceps device 106 may comprise a jaw assembly according to any one of the embodiments disclosed herein or of other type. In accordance with the embodiments described above, biopsy forceps device 106 may comprise first and second jaws 10, 12 configured to cooperatively harvest and retain one or more biopsy samples 104. Collection member 108 may be receivable between jaws 10, 12 of biopsy forceps device 106 as shown in FIG. 15B for collecting (retrieving) the one or more biopsy samples 104 from biopsy forceps device 106. Collection member 108 may comprise adherent portion 110 for contacting with biopsy samples 104 and permitting the collection of biopsy samples 104 from biopsy forceps device 106 upon removal of collection member 108 from between jaws 10, 12 of biopsy forceps device 106.

In some embodiments, jaws 10, 12 of biopsy forceps device 106 may be of a type configured to retain all of the biopsy samples 104 harvested in the same jaw such as jaw assembly 200 (see FIGS. 2A-2C) for example. Jaws 10, 12 of biopsy forceps device 106 may be configured to retain a row of biopsy samples 104 in the order and orientation harvested from the patient. Accordingly, adherent portion 110 may only be disposed on one side of collection member 108. In some embodiments where biopsy samples 104 may be adhered to both jaws 10, 12 of biopsy forceps device 106, it may be desirable to have an adherent portion 110 on both sides of collection member 108. In some embodiments, it may be advantageous to the clinician or the assistant to have an adherent portion 110 on both sides of collection member 108 to eliminate having to determine which side of collection member 108 is adherent and which one is not.

In various embodiments, collection member 108 may have a sheet or plate configuration. In various embodiments, collection member 108 may be substantially flat or may be curved (e.g., concave or convex), or a combination of flat and curved. In some embodiments, collection member 108 may be shaped to fit a pathology processing cartridge. In some embodiments, collection member 108 may comprise cut-out 112 adapted to accommodate part(s) of biopsy forceps device 106 and allow jaws 10, 12 of biopsy forceps device 106 to be at least partially closed with collection member 108 disposed therebetween.

Adherent portion 110 may be configured to come into contact with biopsy samples 104 and cause biopsy samples 104 to adhere thereto so as to cause biopsy samples 104 to be retrieved from biopsy forceps device 106 after jaws 10, 12 of biopsy forceps device 106 have been closed onto collection member 108, opened and removed from collection member 108. In some embodiments, adherent portion 110 may comprise a chemical bonding agent. For example, adherent portion 110 may comprise a suitable epoxy which may be activated by moisture, light, heat or by other means. For example, adherent portion 110 may comprise a suitable epoxy which may be activated by moisture present in biopsy samples 104. In some embodiments, adherent portion 110 may comprise a cyanoacrylate epoxy. In some embodiments, adherent portion 110 may comprise a suitable adhesive (e.g., glue) such as a cyanoacrylate adhesive for example. In some embodiments, adherent portion 110 may be configured to cause one or more biopsy samples adhesion through freeze bonding. In some embodiments, adherent portion 110 may be configured to cause one or more biopsy samples adhesion through dry bonding. In some embodiments, adherent portion 110 may comprise a hydrophilic substance (e.g., gelatin) activated by moisture present in biopsy samples 104.

In some embodiments, adherent portion 110 may comprise a mechanical bonding agent. For example, adherent portion 110 may comprise a suitable surface texture, weave, perforations, spikes, hooks, barbs, divots, slots, windows, slits and/or other sample-adhering features arranged in a grid pattern. In some embodiments, adherent portion 110 may comprise an array of plurality of sample-adhering features. In some embodiments, collection member 108 and adherent portion 110 may be flexible so as to permit some resilient bending of collection member 108 to cause some feature(s) (e.g., slits) to open/close by such bending and grab onto biopsy samples 104. In some embodiments, adherent portion 110 may be configured to cause adherence of biopsy samples 104 to collection member 108 by way of capillary action. For example, adherent portion 110 may comprise features (e.g., small bores, cavities, crevices) formed therein to facilitate such adherence by capillary action.

In some embodiments, adherent portion 110 may comprise a combination of two or more types of bonding agents. For example, adherent portion 110 could comprise a mechanical bonding agent and a chemical bonding agent.

In some embodiments, adherent portion 110 may be configured to cause electrostatic bonding of biopsy samples 104 to collection member 108. In some embodiments, adherent portion 110 may be configured to cause coagulation (e.g., electrocoagulation and/or heat coagulation) bonding of biopsy samples 104 to collection member 108. For example, a thermally-activated adherence (i.e., thermal bonding) of biopsy samples 104 onto collection member 108 could be achieved by having collection member 108 at a different temperature than that of biopsy samples 104 during sample collection in order to cause freezing, drying and/or coagulation of part of biopsy samples 104 when coming in contact with adherent portion 110 and thereby causing adhesion to adherent portion 110.

In some embodiments, adherent portion 110 may be configured to cause adherence of biopsy samples 104 to collection member 108 by way of surface tension. In some embodiments, perforations formed in adherent portion 110 may serve as vacuum ports in communication with vacuum source 114.

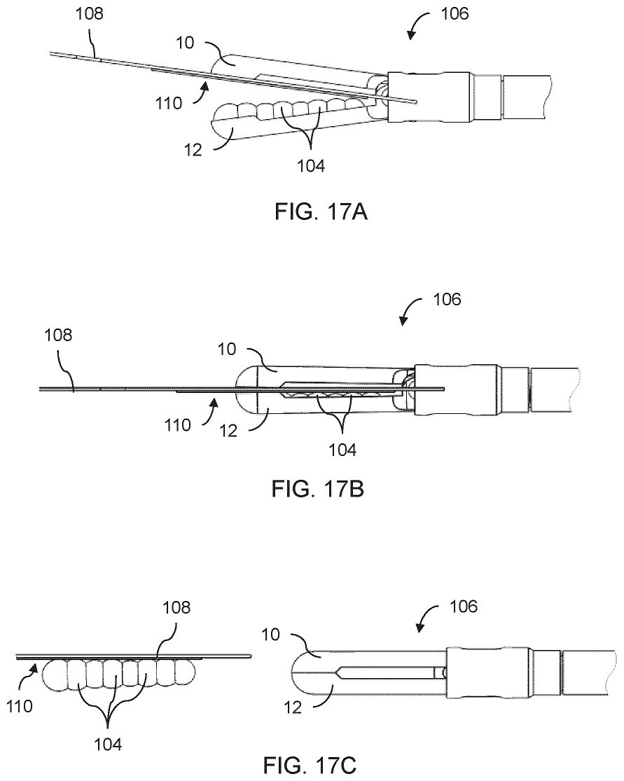

FIGS. 16A-16C graphically illustrate a method for collecting biopsy samples 104 from biopsy forceps device 106 where biopsy samples 104 are retained in upper jaw 10 of biopsy forceps device 106. Similarly, FIGS. 17A-17C graphically illustrate a method for collecting biopsy samples 104 from biopsy forceps device 106 where biopsy samples 104 are retained in lower jaw 12 of biopsy forceps device 106.

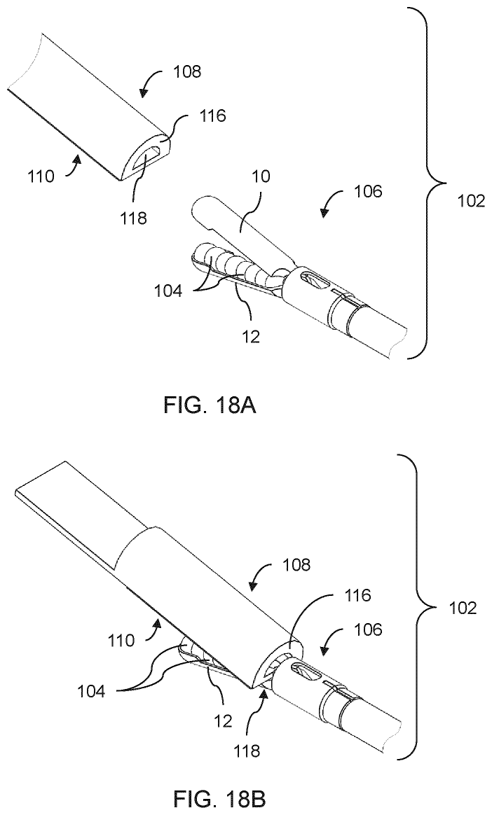

FIGS. 18A and 18B show perspective views of another kit 102 for harvesting one or more biopsy samples 104. Kit 102 may comprise biopsy forceps device 106 and collection member 108. In some embodiments, collection member 108 may comprise one or more guides 116 cooperating with part(s) of biopsy forceps device 106 for constraining a disposition (e.g., position and/or orientation) of biopsy forceps device 106 during the collection of the one or more biopsy samples 104. In some embodiments, guide 116 may define opening 118 for receiving first or second jaw 10, 12 therethrough. Opening 118 may have a shape that substantially matches at least some (e.g., a majority) of a transverse cross-sectional profile of the first or second jaw 10, 12 received therethrough for constraining the orientation of biopsy forceps device 106. For example, collection member 108 may have a tubular configuration where opening 118 defined by guide 116 is an opening to a lumen of tubular collection member 108 in which first or second jaw 10, 12 is received during the collection of the one or more biopsy samples 104. In some embodiments, opening 118 may have a D-shape profile that is slightly larger than a corresponding transverse cross-sectional profile of similar shape of first or second jaw 10, 12.

Adherent portion 110 may be disposed on an outer wall of tubular collection member 108 so that when first jaw 10 is received through opening 118 as shown in FIG. 18B, the adherent portion 110 may be disposed to contact the one or more biopsy samples 104 that are disposed inside of second jaw 12. The use of guide 116 may facilitate the removal of biopsy samples 104 from biopsy forceps device 106 by an operator while retaining the order in which biopsy samples 104 where harvested.

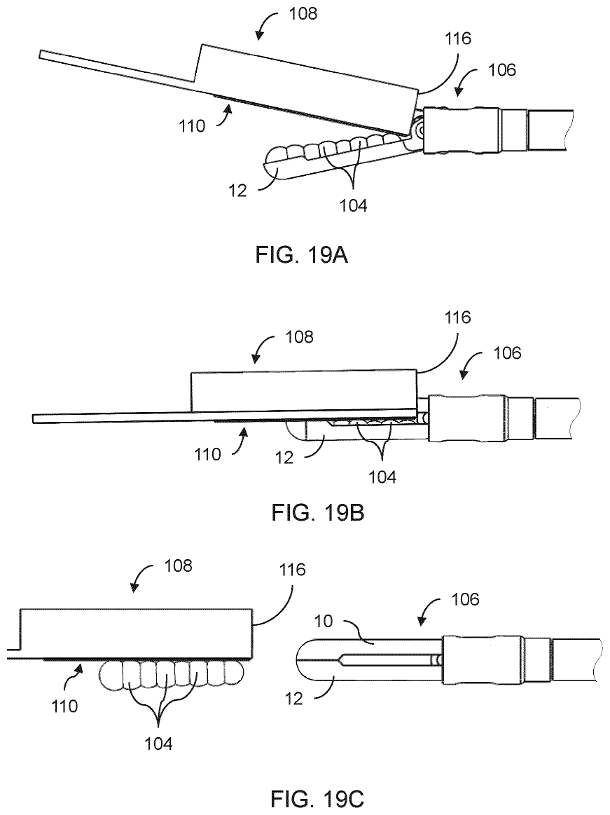

FIGS. 19A-19C graphically illustrate a method for collecting biopsy samples 104 from biopsy forceps device 106 where biopsy samples 104 are retained in lower jaw 12 of biopsy forceps device 106.

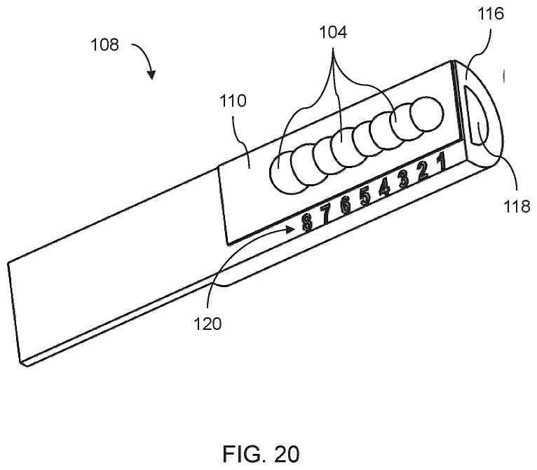

FIG. 20 is a perspective view of the collection member 108 of FIGS. 18A and 18B after biopsy samples 104 have been collected from biopsy forceps device 106 and are being retained by adherent portion 110 thereof. In some embodiments, collection member 108 may comprise one or more labels 120 that are indicative of the order of harvest of biopsy samples 104 to facilitate tracking and further processing or analysis of biopsy samples 104. In various embodiments, label 120 may be textual (e. g., letter(s) and/or number(s)), graphical (e.g., arrow) or may contain a combination of one or more text characters and one or more graphics.

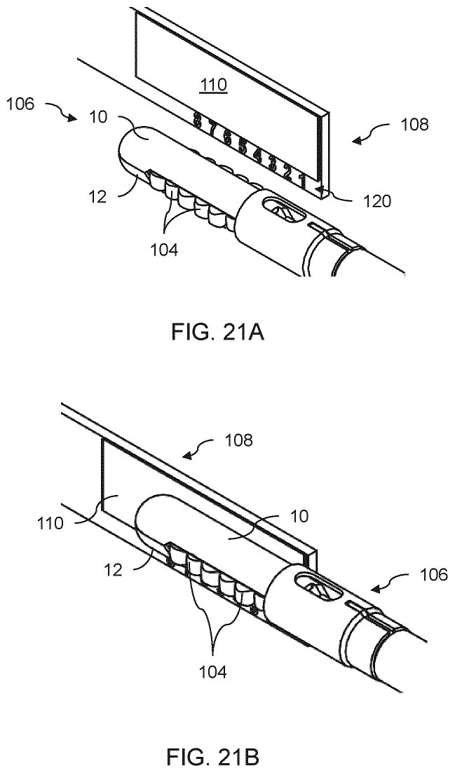

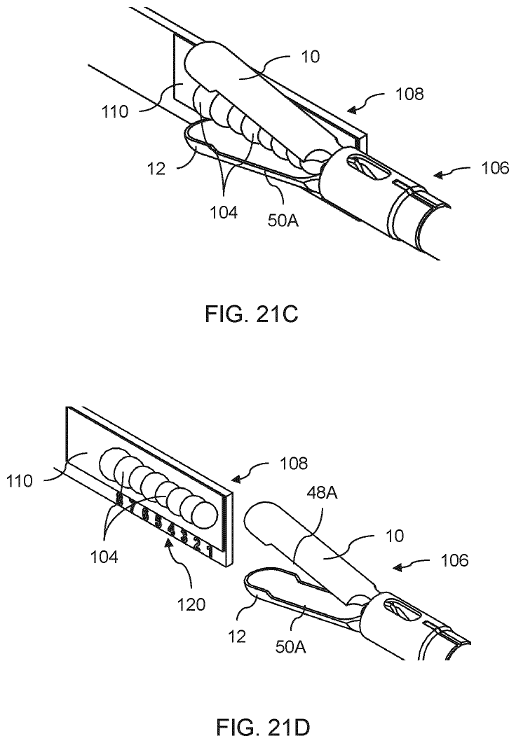

FIGS. 21A-21D are perspective views that graphically illustrate a sequence of steps of another method for collecting biopsy samples 104 from biopsy forceps device 106. In some embodiments, collection member 108 may be configured to contact biopsy samples 104 without necessarily having to be received between first and second jaws 10, 12. For example, adherent portion 110 of collection member 108 may contact portions of biopsy samples 104 that may protrude from first and second jaws 10, 12. For example, adherent portion 110 of collection member 108 may contact portions of biopsy samples 104 that may protrude from between faces 54, 56 of first and second jaws 10, 12. For example, adherent portion 110 of collection member 108 may contact portions of biopsy samples 104 that may protrude from windows 48A and/or windows 50A formed in first and second jaws 10, 12 respectively.

Adherent portion 110 of collection member 108 may, for example, be placed to a side of biopsy forceps device 106 so as to contact protruding portions of biopsy samples 104 and thereby cause adhesion of biopsy samples 104 to adherent portion 110. After such adhesion, first and/or second jaws 10, 12 may be opened to release biopsy samples 104 and collection member 108 (and adherent portion 110) may be withdrawn from biopsy forceps device 106 in order to collect biopsy samples 104 from biopsy forceps device 106. It is understood that biopsy forceps device 106 could instead or in addition be withdrawn from collection member 108 (and adherent portion 110) in order to collect biopsy samples 104 from biopsy forceps device 106. Withdrawal of collection member 108 and biopsy forceps device 106 from each other may include moving adherent portion 110 away from biopsy forceps device 106 and/or moving biopsy forceps device 106 away from adherent portion 110.

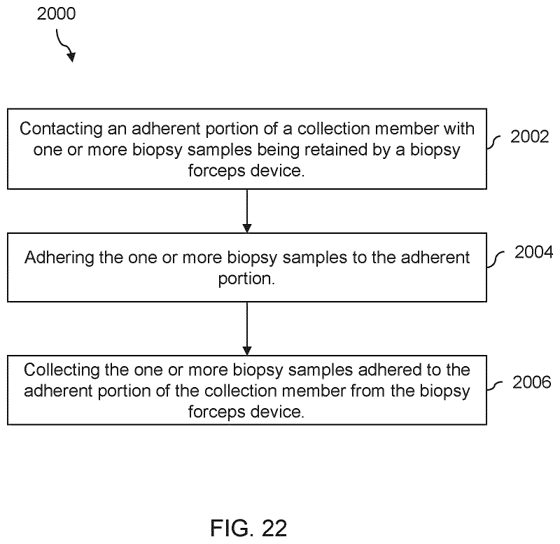

FIG. 22 shows a flowchart of method 2000 in accordance with the graphic illustrations of FIGS. 16A-16C, FIGS. 17A-17C, FIGS. 19A-19C and FIGS. 21A-21C. Method 2000 may be performed using kit 102. Method 2000 may be used to collect (retrieve) one or more biopsy samples 104 from a biopsy forceps device 106 comprising jaws 10, 12 configured to cooperatively harvest and retain one or more biopsy samples 104. Method 2000 may comprise: contacting adherent portion 110 of collection member 108 with the one or more biopsy samples 104 being retained by the biopsy forceps device 106 (see block 2002); adhering the one or more biopsy samples 104 to adherent portion 110 (see block 2004); and collecting the one or more biopsy samples 104 adhered to the adherent portion 110 of collection member 108 from biopsy forceps device 106 (see block 2006).

In some embodiments, contacting adherent portion 110 of collection member 108 with the one or more biopsy samples 104 may comprise receiving collection member 108 between jaws 10, 12 of biopsy forceps device 106. Correspondingly, withdrawing collection member 108 and biopsy forceps device 106 from each other may comprise removing collection member 108 from between jaws 10, 12 to collect the one or more biopsy samples 104 from biopsy forceps device 106.

The use of collection member 108 for collecting biopsy samples 104 from biopsy forceps device 106 may maintain the order and orientation of harvest that was represented in the row of biopsy samples 104 retained in jaws 10, 12 of biopsy forceps device 106. Maintaining the order of harvest may allow biopsy samples 104 to be associated with their respective locations of harvest within the body of the patient and may be important to the analysis of biopsy samples 104 and to the diagnosis and treatment of the patient. Maintaining the order of harvest may also be important to the analysis of biopsy samples 104 and to the diagnosis and treatment of the patient.

As shown in FIGS. 16C, 17C and 19C, the row of biopsy samples 104 may be transferred onto collection member 108 without changing the order and orientation in which biopsy samples 104 were harvested. After the collection of biopsy samples 104, collection member 108 and biopsy samples 104 may together be sent to a lab for processing and histopathological analysis as a cluster of samples 104 positioned in their order of harvest. In some situations, collection member 108 may also fit in the pathology processing cartridge, or be a part of it. The use of collection member 108 may, in some situations, reduce or eliminate the risk of damaging biopsy samples 104 during the collection of biopsy samples 104 from jaw(s) 10, 12 of biopsy forceps device 106.

In some embodiments, method 2000 may comprise clamping collection member 108 between jaws 10, 12 to adhere the one or more biopsy samples 104 to collection member 108. Method 2000 may also comprise unclamping collection member 108 before removing collection member 108 from between jaws 10, 12. Removal of collection member 108 from between jaws 10, 12 may be done manually. Removal of collection member 108 from between jaws 10, 12 could be done by shaking off collection member 108 along with biopsy samples 104 from the open jaws 10, 12 and into a jar containing fixative fluid.

In some embodiments, adhering the one or more biopsy samples 104 to collection member 108 may comprise chemically bonding the one or more biopsy samples 104 to collection member 108.

In some embodiments, adhering the one or more biopsy samples 104 to collection member 108 may comprise bonding the one or more biopsy samples 104 to collection member 108 using an epoxy.

In some embodiments, adhering the one or more biopsy samples 104 to collection member 108 may comprise mechanically bonding the one or more biopsy samples 104 to collection member 108.

In some embodiments, adhering the one or more biopsy samples 104 to collection member 108 may comprise electrostatically bonding the one or more biopsy samples 104 to collection member 108.

In some embodiments, method 2000 may comprise bonding the one or more biopsy samples 104 to collection member 108 using coagulation (e.g., electrocoagulation and/or heat coagulation) bonding.

In some embodiments, method 2000 may comprise bonding the one or more biopsy samples 104 to collection member 108 using vacuum source 114.

In some embodiments, method 2000 may comprise bonding the one or more biopsy samples 104 to collection member 108 using two or more bonding approaches. In some embodiments, method 2000 may comprise bonding the one or more biopsy samples 104 to collection member 108 using two or more bonding approaches disclosed herein.

The above description is meant to be exemplary only, and one skilled in the relevant arts will recognize that changes may be made to the embodiments described without departing from the scope of the invention disclosed. The present disclosure may be embodied in other specific forms without departing from the subject matter of the claims. The present disclosure is intended to cover and embrace all suitable changes in technology. Modifications which fall within the scope of the present invention will be apparent to those skilled in the art, in light of a review of this disclosure, and such modifications are intended to fall within the appended claims. Also, the scope of the claims should not be limited by the preferred embodiments set forth in the examples, but should be given the broadest interpretation consistent with the description as a whole.

1. A jaw assembly for a biopsy forceps device, the assembly comprising:
first and second jaws pivotable about a common pivot axis, the first and second jaws being pivotally movable between respective open and closed positions and configured to cooperatively harvest and retain one or more biopsy samples by actuation of the first and second jaws between their respective open and closed positions;
one or more actuation members for actuating the first and second jaws between their respective open and closed positions; and
one or more guides configured to urge one or more of the first and second jaws toward their respective closed positions when the one or more jaws are actuated toward their respective closed positions using the one or more actuation members.

2. The assembly as defined in claim 1, wherein the one or more actuation members are configured to cause translation movement of the first and second jaws relative to the one or more guides.

3. The assembly as defined in claim 1, wherein the one or more actuation members are configured to cause movement of the first and second jaws toward the one or more guides when the first and second jaws are actuated toward their respective closed positions.

4. The assembly as defined in claim 1, wherein the one or more actuation members comprise a common actuation wire configured to actuate both the first and second jaws.

5. The assembly as defined in claim 1, wherein the one or more actuation members comprise respective actuation wires for actuating the first and second jaws.

6. The assembly as defined in claim 5, wherein the actuation wires are configured to be actuated in unison to cause opening and closing of the first and second jaws.

7. The assembly as defined in claim 5, wherein the actuation wires are connected to respective ones of the first and second jaws at respective offset distances from the common pivot axis.

8. The assembly as defined in claim 1, wherein the one or more guides comprise an aperture into which a portion of the first and second jaws is progressively received during closing of the first and second jaws.

9. The assembly as defined in claim 8, wherein the aperture is defined by an urging surface configured to engage with respective radially-outer surfaces of the first and second jaws during closing of the first and second jaws.

10. The assembly as defined in claim 1, wherein the one or more guides comprise a sleeve.

11. The assembly as defined in claim 1, wherein the first and second jaws cooperatively define a sample-retaining volume configured to retain a plurality of biopsy samples in an order of harvest.

12. (canceled)

13. The assembly as defined in claim 11, wherein the one or more guides comprise a sleeve into which a portion of the first and second jaws is progressively received during closing of the first and second jaws.

14. The assembly as defined in claim 13, wherein the first and second jaws are configured so that at least a majority of the sample-retaining volume remains outside of the sleeve when the first and second jaws are closed.

15. The assembly as defined in claim 1, wherein the first and second jaws are pivotally coupled to a common pivot member.

16. The assembly as defined in claim 1, wherein the first and second jaws are substantially rigid.

17. The assembly as defined in claim 1, wherein:
the one or more guides comprise a sleeve;
the first and second jaws are pivotally coupled to a common pivot member;
opposite ends of the common pivot member are engaged with the sleeve; and
the common pivot member is translatable relative to the sleeve during closing of the first and second jaws.

18. The assembly as defined in claim 17, wherein the sleeve comprises diametrically opposed slots engaging respective opposite ends of the common pivot member.

19. The assembly as defined in claim 1, wherein:
the first and second jaws define a sample-retaining volume having an elongated shape having a central longitudinal axis; and
the one or more guides define a maximum radially-outer dimension that is the same or less than a maximum radially-outer dimension defined by the first and second jaws when the first and second jaws are closed.

20. The assembly as defined in claim 1, wherein:
the one or more guides comprise a sleeve having a central axis;
the first and second jaws each comprise a radially-outer surface and a transition surface portion extending between two regions of the radially-outer surface having different radially-outer dimensions relative to the central axis of the sleeve; and
the sleeve comprises one or more urging surfaces configured to engage with the transition surface portions of the first and second jaws during closing of the first and second jaws.

21. The assembly as defined in claim 1, wherein:
the one or more guides comprise a sleeve having a central axis;
the first and second jaws each comprise a radially-outer surface and a transition surface portion extending radially inwardly from the radially-outer surface relative to the central axis of the sleeve; and
the sleeve comprises one or more urging surfaces configured to engage with the transition surface portions of the first and second jaws during closing of the first and second jaws.

22. The assembly as defined in claim 20, wherein the transition surface portions are substantially transverse to the central axis of the sleeve.

23. The assembly as defined in claim 20, wherein the transition surface portions are substantially oblique to the central axis of the sleeve.

24. The assembly as defined in claim 1, wherein the common pivot axis has a variable position relative to the one or more guides.

25. The assembly as defined in claim 1, wherein the first jaw has a first rocking surface for interfacing with a second rocking surface of the second jaw during pivotal movement of the first and second jaws.

26. The assembly as defined in claim 25, wherein the common pivot axis is disposed at an interface between the first rocking surface and the second rocking surface.

27. The assembly as defined in claim 1, wherein the first and second jaws are slidingly engaged to respective parallel guide pins.

28. The assembly as defined in claim 27, wherein the guide pins are secured to the one or more guides.

29. The assembly as defined in claim 1, wherein the first and second jaws are slidingly engaged to a common guide pin.

30. The assembly as defined in claim 29, wherein the common guide pin has a rectangular transverse cross-sectional profile.

31. The assembly as defined in claim 29, wherein a longitudinal axis of the common guide pin is transverse to the common pivot axis.

32. The assembly as defined in claim 1, wherein:
the one or more guides comprise a sleeve having a central axis;
the first and second jaws are slidingly engaged to a common guide pin;
the common guide pin has a rectangular transverse cross-sectional profile;
a dimension of the transverse cross-sectional profile along the central axis of the sleeve is greater than a dimension of the transverse cross-sectional profile transverse to the central axis of the sleeve.

33. The assembly as defined in claim 29, wherein the common guide pin is secured to the one or more guides.

34. The assembly as defined in claim 1, wherein:
the one or more guides comprise a sleeve having a central axis; and
the assembly comprises a guide insert for guiding the movement of the first and second jaws, the guide insert being disposed inside of the sleeve, the guide insert comprising one or more resilient features for engagement with one or more corresponding sleeve features and for retention of the guide insert inside of the sleeve, the one or more resilient features being resiliently movable in a radially inward direction relative to the central axis of the sleeve.

35. The assembly as defined in claim 34, wherein the one or more sleeve features comprise respective receptacles formed in the sleeve for receiving the corresponding one or more resilient features of the guide insert, the one or more resilient features of the guide insert extending radially outwardly into the corresponding one or more receptacles.

36. The assembly as defined in claim 35, wherein the guide insert comprises one or more cutouts to facilitate resilient radially inward movement of the one or more resilient features and thereby facilitate insertion of the guide insert into the sleeve during assembly.

37. The assembly as defined in claim 34, wherein the guide insert comprises one or more stoppers for limiting an opening movement of one or more of the first and second jaws.

38. The assembly as defined in claim 34, wherein the guide insert comprises a surface defining a proximal end of a sample-retaining volume cooperatively defined by the first and second jaws.

39. A jaw assembly for an endoscopic surgical tool, the assembly comprising:
first and second jaws pivotable about a common pivot axis, the first and second jaws being pivotally movable between respective open and closed positions;
one or more actuation members for actuating the first and second jaws between their respective open and closed positions; and
one or more guides configured to urge one or more of the first and second jaws toward their respective closed positions when the one or more jaws are actuated toward their respective closed positions using the one or more actuation members.

40.-125. (canceled)
